SEQUENCE LISTING (17 sequences)

SEQ ID NO: 1 - DNA, 1680 bp, Mus musculus
tgaaaagatagaataaatggcctcgtgccgaattcggcacgagccgaggcgagggcctgcatggcgcggccagcgctgctgggcgagctgttggtgctgctactgtggaccgccaccgtgggccaagttgccgcggccacagaagttcagccacctgtgacgaatttgagcgtctctgtcgaaaatctctgcacgataatatggacgtggagtcctcctgaaggagccagtccaaattgcactctcagatattttagtcactttgatgaccaacaggataagaaaattgctccagaaactcatcgtaaagaggaattacccctggatgagaaaatctgtctgcaggtgggctctcagtgtagtgccaatgaaagtgagaagcctagccctttggtgaaaaagtgcatctcaccccctgaaggtgatcctgagtccgctgtgactgagctcaagtgcatttggcataacctgagctatatgaagtgttcctggctccctggaaggaatacaagccctgacacacactatactctgtactattggtacagcagcctggagaaaagtcgtcaatgtgaaaacatctatagagaaggtcaacacattgcttgttcctttaaattgactaaagtggaacctagttttgaacatcagaacgttcaaataatggtcaaggataatgctgggaaaattaggccatcctgcaaaatagtgtctttaacttcctatgtgaaacctgatcctccacatattaaacatcttctcctcaaaaatggtgccttattagtgcagtggaagaatccacaaaattttagaagcagatgcttaacttatgaagtggaggtcaataatactcaaaccgaccgacataatattttagaggttgaagaggacaaatgccagaattccgaatctgatagaaacatggagggtacaagttgtttccaactccctggtgttcttgccgacgctgtctacacagtcagagtaagagtcaaaacaaacaagttatgctttgatgacaacaaactgtggagtgattggagtgaagcacagagtataggtaaggagcaaaactccaccttctacaccaccatgttactcaccattccagtctttgtcgcagtggcagtcataatcctccttttttacctgaaaaggcttaagatcattatatttcctccaattcctgatcctggcaagatttttaaagaaatgtttggagaccagaatgatgataccctgcactggaagaagtatgacatctatgagaaacaatccaaagaagaaacggattctgtagtgctgatagaaaacctgaagaaagcagctccttgatggggagaagtgatttctttcttgccttcaatgtgaccctgtgaagatttattgcattctccatttgttatctgggggacttgttaaatagaaactgaaactactcttgaaaaacaggcagctcctaagagccacaggtcttgatgtgacttttgcattgaaaacccaaacccaaaggagctccttccaagaaaagcaagagttcttctcgttccttgttccaatccctaaaagcagatgttttgccaaatccccaaactagaggacaaagacaaggggacaatgaccatcaattcatctaatcaggaattgtgatggcttcctaaggaatctctgcttgctctg

SEQ ID NO: 2 - protein, 424 aa, Mus musculus
MARPALLGELLVLLLWTATVGQVAAATEVQPPVTNLSVSVENLCTIIWTWSPPEGASPNCTLRYFSHFDDQQDKKIAPETHRKEELPLDEKICLQVGSQCSANESEKPSPLVKKCISPPEGDPESAVTELKCIWHNLSYMKCSWLPGRNTSPDTHYTLYYWYSSLEKSRQCENIYREGQHIACSFKLTKVEPSFEHQNVQIMVKDNAGKIRPSCKIVSLTSYVKPDPPHIKHLLLKNGALLVQWKNPQNFRSRCLTYEVEVNNTQTDRHNILEVEEDKCQNSESDRNMEGTSCFQLPGVLADAVYTVRVRVKTNKLCFDDNKLWSDWSEAQSIGKEQNSTFYTTMLLTIPVFVAVAVIILLFYLKRLKIIIFPPIPDPGKIFKEMFGDQNDDTLHWKKYDIYEKQSKEETDSVVLIENLKKAAP

SEQ ID NO: 3 - DNA, 1567 bp, Mus musculus
ggcacgagggagaggaggagggaaagatagaaagagagagagaaagattgcttgctacccctgaacagtgacctctctcaagacagtgctttgctcttcacgtataaggaaggaaaacagtagagattcaatttagtgtctaatgtggaaaggaggacaaagaggtcttgtgataactgcctgtgataatacatttcttgagaaaccatattattgagtagagctttcagcacactaaatcctggagaaatggcttttgtgcatatcagatgcttgtgtttcattcttctttgtacaataactggctattctttggagataaaagttaatcctcctcaggattttgaaatattggatcctggattacttggttatctctatttgcaatggaaacctcctgtggttatagaaaaatttaagggctgtacactagaatatgagttaaaataccgaaatgttgatagcgacagctggaagactataattactaggaatctaatttacaaggatgggtttgatcttaataaaggcattgaaggaaagatacgtacgcatttgtcagagcattgtacaaatggatcagaagtacaaagtccatggatagaagcttcttatgggatatcagatgaaggaagtttggaaactaaaattcaggacatgaagtgtatatattataactggcagtatttggtctgctcttggaaacctggcaagacagtatattctgataccaactataccatgtttttctggtatgagggcttggatcatgccttacagtgtgctgattacctccagcatgatgaaaaaaatgttggatgcaaactgtccaacttggactcatcagactataaagatttttttatctgtgttaatggatcttcaaagttggaacccatcagatccagctatacagtttttcaacttcaaaatatagttaaaccattgccaccagaattccttcatattagtgtggagaattccattgatattagaatgaaatggagcacacctggaggacccattccaccaaggtgttacacttatgaaattgtgatccgagaagacgatatttcctgggagtctgccacagacaaaaacgatatgaagttgaagaggagagcaaatgaaagtgaagacctatgcttttttgtaagatgtaaggtcaatatatattgtgcagatgatggaatttggagcgaatggagtgaagaggaatgttgggaaggttacacagggccagactcaaagattattttcatagtaccagtttgtcttttctttatattccttttgttacttctttgccttattgtggagaaggaagaacctgaacccacattgagcctccatgtggatctgaacaaagaagtgtgtgcttatgaagataccctctgttaaaccaccaatttcttgacatagagccagccagcaggagtcatattaaactcaatttctcttaaaatttcgaatacatcttcttgaaaatcagtgtttgtcctaatagtgttgggtttttgactaaagtgctggatatatatctccaaaaaaaaaaaaaaaaaaaaaa

SEQ ID NO: 4 - protein, 383 aa, Mus musculus
MAFVHIRCLCFILLCTITGYSLEIKVNPPQDFEILDPGLLGYLYLQWKPPVVIEKFKGCTLEYELKYRNVDSDSWKTIITRNLIYKDGFDLNKGIEGKIRTHLSEHCTNGSEVQSPWIEASYGISDEGSLETKIQDMKCIYYNWQYLVCSWKPGKTVYSDTNYTMFFWYEGLDHALQCADYLQHDEKNVGCKLSNLDSSDYKDFFICVNGSSKLEPIRSSYTVFQLQNIVKPLPPEFLHISVENSIDIRMKWSTPGGPIPPRCYTYEIVIREDDISWESATDKNDMKLKRRANESEDLCFFVRCKVNIYCADDGIWSEWSEEECWEGYTGPDSKIIFIVPVCLFFIFLLLLLCLIVEKEEPEPTLSLHVDLNKEVCAYEDTLC

SEQ ID NO: 5 - DNA, 1382 bp, Homo sapiens
cggatgaaggctatttgaagtcgccataacctggtcagaagtgtgcctgtcggcggggagagaggcaatatcaaggttttaaatctcggagaaatggctttcgtttgcttggctatcggatgcttatatacctttctgataagcacaacatttggctgtacttcatcttcagacaccgagataaaagttaaccctcctcaggattttgagatagtggatcccggatacttaggttatctctatttgcaatggcaacccccactgtctctggatcattttaaggaatgcacagtggaatatgaactaaaataccgaaacattggtagtgaaacatggaagaccatcattactaagaatctacattacaaagatgggtttgatcttaacaagggcattgaagcgaagatacacacgcttttaccatggcaatgcacaaatggatcagaagttcaaagttcctgggcagaaactacttattggatatcaccacaaggaattccagaaactaaagttcaggatatggattgcgtatattacaattggcaatatttactctgttcttggaaacctggcataggtgtacttcttgataccaattacaacttgttttactggtatgagggcttggatcatgcattacagtgtgttgattacatcaaggctgatggacaaaatataggatgcagatttccctatttggaggcatcagactataaagatttctatatttgtgttaatggatcatcagagaacaagcctatcagatccagttatttcacttttcagcttcaaaatatagttaaacctttgccgccagtctatcttacttttactcgggagagttcatgtgaaattaagctgaaatggagcatacctttgggacctattccagcaaggtgttttgattatgaaattgagatcagagaagatgatactaccttggtgactgctacagttgaaaatgaaacatacaccttgaaaacaacaaatgaaacccgacaattatgctttgtagtaagaagcaaagtgaatatttattgctcagatgacggaatttggagtgagtggagtgataaacaatgctgggaaggtgaagacctatcgaagaaaactttgctacgtttctggctaccatttggtttcatcttaatattagttatatttgtaaccggtctgcttttgcgtaagccaaacacctacccaaaaatgattccagaatttttctgtgatacatgaagactttccatatcaagagacatggtattgactcaacagtttccagtcatggccaaatgttcaatatgagtctcaataaactgaatttttcttgcgaaaaaaaaaaaaaaaaaaaaaaaaaaaaaaaaaaaaaaaaaaaaaaaaaa

SEQ ID NO: 6 - protein, 380 aa, Homo sapiens
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT

SEQ ID NO: 7 - DNA, 2802 bp, Homo sapiens
agctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagcccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagggaggcagagcgggtcccactgtccccacactggcccaggctgtgcaggtgtgcctgggccccctagggtggggctcagccaggggctgccctcggcagggtgggggatttgccagcgtggccctccctccagcagcacctgccctgggctgggccacgggaagccctaggagcccctggggacagacacacagcccctgcctctgtaggagactgtcctgttctgtgagcgcccctgtcctcccgacctccatgcccactcgggggcatgcctagtccatgtgcgtagggacaggccctccctcacccatctacccccacggcactaacccctggctgccctgcccagcctcgcacccgcatggggacacaaccgactccggggacatgcactctcgggccctgtggagggactggtgcagatgcccacacacacactcagcccagacccgttcaacaaaccccgcactgaggttggccggccacacggccaccacacacacacgtgcacgcctcacacacggagcctcacccgggcgaactgcacagcacccagaccagagcaaggtcctcgcacacgtgaacactcctcggacacaggcccccacgagccccacgcggcacctcaaggcccacgagcctctcggcagcttctccacatgctgacctgctcagacaaacccagccctcctctcacaagggtgcccctgcagccgccacacacacacaggggatcacacaccacgtcacgtccctggccctggcccacttcccagtgccgcccttccctgcagacggatcc

SEQ ID NO: 8 - protein, 330 aa, Homo sapiens [sequence omitted]

SEQ ID NO: 9 - DNA, 20 bp, Artificial Sequence
  Description of Artificial Sequenceoligonucleotide primer
atagttaaaccattgccacc

SEQ ID NO: 10 - DNA, 20 bp, Artificial Sequence
  Description of Artificial Sequenceoligonucleotide primer
ctccattcgctccaaattcc

SEQ ID NO: 11 - DNA, 21 bp, Artificial Sequence
  Description of Artificial Sequenceoligonucleotide primer
agtctatcttacttttactcg

SEQ ID NO: 12 - DNA, 22 bp, Artificial Sequence
  Description of Artificial Sequenceoligonucleotide primer
catctgagcaataaatattcac

SEQ ID NO: 13 - protein, 565 aa, Homo sapiens
MKFLVNVALVFMVVYISYIYATEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 187 aa, Homo sapiens
MVRPLNCIVAVSQNMGIGKNGDLPWPPLRNEFKYFQRMTTTSSVEGKQNLVIMGRKTWFSIPEKNRPLKDRINIVLSRELKEPPRGAHFLAKSLDDALRLIEQPELASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 15 - DNA, 6802 bp, Artificial Sequence
  Description of Artificial Sequenceplasmid expression vector
catatgcggtgtgaaataccgcacagatgcgtaaggagaaaataccgcatcaggcgtactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcagcccaagctctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgactctagcgcaccaccatgaaattcttagtcaacgttgcccttgtttttatggtcgtgtacatttcttacatctatgcgaccgagataaaagttaaccctcctcaggattttgagatagtggatcccggatacttaggttatctctatttgcaatggcaacccccactgtctctggatcattttaaggaatgcacagtggaatatgaactaaaataccgaaacattggtagtgaaacatggaagaccatcattactaagaatctacattacaaagatgggtttgatcttaacaagggcattgaagcgaagatacacacgcttttaccatggcaatgcacaaatggatcagaagttcaaagttcctgggcagaaactacttattggatatcaccacaaggaattccagaaactaaagttcaggatatggattgcgtatattacaattggcaatatttactctgttcttggaaacctggcataggtgtacttcttgataccaattacaacttgttttactggtatgagggcttggatcatgcattacagtgtgttgattacatcaaggctgatggacaaaatataggatgcagatttccctatttggaggcatcagactataaagatttctatatttgtgttaatggatcatcagagaacaagcctatcagatccagttatttcacttttcagcttcaaaatatagttaaacctttgccgccagtctatcttacttttactcgggagagttcatgtgaaattaagctgaaatggagcatacctttgggacctattccagcaaggtgttttgattatgaaattgagatcagagaagatgatactaccttggtgactgctacagttgaaaatgaaacatacaccttgaaaacaacaaatgaaacccgacaattatgctttgtagtaagaagcaaagtgaatatttattgctcagatgacggaatttggagtgagtggagtgataaacaatgctgggaaggtgaagacctatcgaagaaaactcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagtgaattaattcggcgcgccaaattctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattgctcgagccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcattttttataagaccatgggacttttgctggctttagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccggccaacggtctggtgacccggctgcgagagctcggtgtacctgagacgcgagtaagcccttgagtcaaagacgtagtcgttgcaagtccgcaccaggtactgatcatcgatgctagaccgtgcaaaaggagagcctgtaagcgggcactcttccgtggtctggtggataaattcgcaagggtatcatggcggacgaccggggttcgaaccccggatccggccgtccgccgtgatccatccggttaccgcccgcgtgtcgaacccaggtgtgcgacgtcagacaacgggggagcgctccttttggcttccttccaggcgcggcggctgctgcgctagcttttttggcgagctcgaattaattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcac

SEQ ID NO: 16 - DNA, 6802 bp, Artificial Sequence
  Description of Artificial Sequenceplasmid expression vector
gtatacgccacactttatggcgtgtctacgcattcctcttttatggcgtagtccgcatgactcagtaatccctgaaaggttacccaaaacgggtcatgtattccagttatccccacttagttgtcctttcagggtaacctcggttcatgtgactcagttatccctgaaaggtaacccaaaacgggtcatgttttccagttatcccccactcagttacccaaaaagggtaataaccgtgcatgtattccagttatccccactcagtaacccaaaaaggtcggttaaattaattttgcggtacatgaaagggtggtaactgcagttacccgataactttgattacgttgcactggaaatttgccatgaaagggtatcgactaattaccctttcatggcaagagctcggttatgtgcagttacccttcactttcccgtcggttttgcattgtggcggggccaaaaggggacctttaaggtataaccgtgcgtaagataaccgactcgacgcaagatgcacccatattctccgcgctggtcgcagccatggcagcgtcagaagccagactggtggcatcttgcgtctcgaggagcgacgtcgggttcgagacaacccgagcgccaactcctgtttgagaagcgccagaaaggtcatgagaacctagcctttgggcagccggaggcttgccatgaggcggtggctccctggactcgctcaggcgtagctggcctagccttttggagagctgacaaccccactcatgagggagagttttcgcccgtactgaagacgcgattctaacagtcaaaggtttttgctcctcctaaactataagtggaccgggcgccactacggaaactcccaccggcgcaggtagaccagtcttttctgttagaaaaacaacagttcgaactccacaccgtccgaactctagaccggtatgtgaactcactgttactgtaggtgaaacggaaagagaggtgtccacaggtgagggtccaggttgacgtccagctgagatcgcgtggtggtactttaagaatcagttgcaacgggaacaaaaataccagcacatgtaaagaatgtagatacgctggctctattttcaattgggaggagtcctaaaactctatcacctagggcctatgaatccaatagagataaacgttaccgttgggggtgacagagacctagtaaaattccttacgtgtcaccttatacttgattttatggctttgtaaccatcactttgtaccttctggtagtaatgattcttagatgtaatgtttctacccaaactagaattgttcccgtaacttcgcttctatgtgtgcgaaaatggtaccgttacgtgtttacctagtcttcaagtttcaaggacccgtctttgatgaataacctatagtggtgttccttaaggtctttgatttcaagtcctatacctaacgcatataatgttaaccgttataaatgagacaagaacctttggaccgtatccacatgaagaactatggttaatgttgaacaaaatgaccatactcccgaacctagtacgtaatgtcacacaactaatgtagttccgactacctgttttatatcctacgtctaaagggataaacctccgtagtctgatatttctaaagatataaacacaattacctagtagtctcttgttcggatagtctaggtcaataaagtgaaaagtcgaagttttatatcaatttggaaacggcggtcagatagaatgaaaatgagccctctcaagtacactttaattcgactttacctcgtatggaaaccctggataaggtcgttccacaaaactaatactttaactctagtctcttctactatgatggaaccactgacgatgtcaacttttactttgtatgtggaacttttgttgtttactttgggctgttaatacgaaacatcattcttcgtttcacttataaataacgagtctactgccttaaacctcactcacctcactatttgttacgacccttccacttctggatagcttcttttgagggtttagaacactgttttgagtgtgtacgggtggcacgggtcgtggacttgaggacccccctggcagtcagaaggagaaggggggttttgggttcctgtgggagtactagagggcctggggactccagtgtacgcaccaccacctgcactcggtgcttctgggactccagttcaagttgaccatgcacctgccgcacctccacgtattacggttctgtttcggcgccctcctcgtcatgttgtcgtgcatggcacaccagtcgcaggagtggcaggacgtggtcctgaccgacttaccgttcctcatgttcacgttccagaggttgtttcgggagggtcaggggtagctcttttggtagaggtttcggtttcccgtcggggctcttggtgtccacatgtgggacgggggtagggccctcctctactggttcttggtccagtcggactggacggaccagtttccgaagatagggtcgctgtagcggcacctcaccctctcgttacccgtcggcctcttgttgatgttctggtgcggagggcacgacctgaggctgccgaggaagaaggagatatcgttcgagtggcacctgttctcgtccaccgtcgtccccttgcagaagagtacgaggcactacgtactccgagacgtgttggtgatgtgcgtcttctcggagagggacaggggcccatttactcacttaattaagccgcgcggtttaagattgcaatgaccggcttcggcgaaccttattccggccacacgcaaacagatatacaataaaaggtggtataacggcagaaaaccgttacactcccgggcctttggaccgggacagaagaactgctcgtaaggatccccagaaaggggagagcggtttccttacgttccagacaacttacagcacttccttcgtcaaggagaccttcgaagaacttctgtttgttgcagacatcgctgggaaacgtccgtcgccttggggggtggaccgctgtccacggagacgccggttttcggtgcacatattctatgtggacgtttccgccgtgttggggtcacggtgcaacactcaacctatcaacacctttctcagtttaccgagaggagttcgcataagttgttccccgacttcctacgggtcttccatggggtaacataccctagactagaccccggagccacgtgtacgaaatgtacacaaatcagctccaattttttgcagatccggggggcttggtgcccctgcaccaaaaggaaactttttgtgctaacgagctcggtagtaccaagctggtaacttgacgtagcagcggcacagggttttatacccctaaccgttcttgcctctggatgggaccggaggcgagtccttgctcaagttcatgaaggtttcttactggtgttggagaagtcaccttccatttgtcttagaccactaatacccatccttttggaccaagaggtaaggactcttcttagctggaaatttcctgtcttaattatatcaagagtcatctcttgagtttcttggtggtgctcctcgagtaaaagaacggttttcaaacctactacggaattctgaataacttgttggccttaaccgttcatttcatctgtaccaaacctatcagcctccgtcaagacaaatggtccttcggtacttagttggtccggtggagtctgagaaacactgttcctagtacgtccttaaactttcactgtgcaaaaagggtctttaactaaacccctttatatttgaagagggtcttatgggtccgcaggagagactccaggtcctcctttttccgtagttcatattcaaacttcagatgctcttctttctgattgtccttctacgaaagttcaagagacgaggggaggatttcgatacgtaaaaaatattctggtaccctgaaaacgaccgaaatctagtattagtcggtatggtgtaaacatctccaaaatgaacgaaattttttggagggtgtggagggggacttggactttgtattttacttacgttaacaacaacaattgaacaaataacgtcgaatattaccaatgtttatttcgttatcgtagtgtttaaagtgtttatttcgtaaaaaaagtgacgtaagatcaacaccaaacaggtttgagtagttacatagaatagtacagacctaggggccggttgccagaccactgggccgacgctctcgagccacatggactctgcgctcattcgggaactcagtttctgcatcagcaacgttcaggcgtggtccatgactagtagctacgatctggcacgttttcctctcggacattcgcccgtgagaaggcaccagaccacctatttaagcgttcccatagtaccgcctgctggccccaagcttggggcctaggccggcaggcggcactaggtaggccaatggcgggcgcacagcttgggtccacacgctgcagtctgttgccccctcgcgaggaaaaccgaaggaaggtccgcgccgccgacgacgcgatcgaaaaaaccgctcgagcttaattaagacgtaattacttagccggttgcgcgcccctctccgccaaacgcataacccgcgagaaggcgaaggagcgagtgactgagcgacgcgagccagcaagccgacgccgctcgccatagtcgagtgagtttccgccattatgccaataggtgtcttagtcccctattgcgtcctttcttgtacactcgttttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagttacgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcctgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttaatttttacttcaaaatttagttagatttcatatatactcatttgaaccagactgtcaatggttacgaattagtcactccgtggatagagtcgctagacagataaagcaagtaggtatcaacggactgaggggcagcacatctattgatgctatgccctcccgaatggtagaccggggtcacgacgttactatggcgctctgggtgcgagtggccgaggtctaaatagtcgttatttggtcggtcggccttcccggctcgcgtcttcaccaggacgttgaaataggcggaggtaggtcagataattaacaacggcccttcgatctcattcatcaagcggtcaattatcaaacgcgttgcaacaacggtaacgatgtccgtagcaccacagtgcgagcagcaaaccataccgaagtaagtcgaggccaagggttgctagttccgctcaatgtactagggggtacaacacgttttttcgccaatcgaggaagccaggaggctagcaacagtcttcattcaaccggcgtcacaatagtgagtaccaataccgtcgtgacgtattaagagaatgacagtacggtaggcattctacgaaaagacactgaccactcatgagttggttcagtaagactcttatcacatacgccgctggctcaacgagaacgggccgcagttatgccctattatggcgcggtgtatcgtcttgaaattttcacgagtagtaaccttttgcaagaagccccgcttttgagagttcctagaatggcgacaactctaggtcaagctacattgggtgagcacgtgggttgactagaagtcgtagaaaatgaaagtggtcgcaaagacccactcgtttttgtccttccgttttacggcgttttttcccttattcccgctgtgcctttacaacttatgagtatgagaaggaaaaagttataataacttcgtaaatagtcccaataacagagtactcgcctatgtataaacttacataaatctttttatttgtttatccccaaggcgcgtgtaaaggggcttttcacggtggactgcagattctttggtaataatagtactgtaattggatatttttatccgcatagtgctccgggaaagcagagcgcgcaaagccactactgccacttttggagactgtgtacgtcgagggcctctgccagtgtcgaacagacattcgcctacggccctcgtctgttcgggcagtcccgcgcagtcgcccacaaccgcccacagccccgaccgaattgatacgccgtagtctcgtctaacatgactctcacgtg

SEQ ID NO: 17 - protein, 132 aa, Homo sapiens
MALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

(19) United States
(12) Patent Application Publication
(10) Pub. No.: US 2005/0118683 A1
(43) Pub. Date: Jun. 2, 2005

(54) Method for producing a polypeptide

(76) Inventors: Clive R. Wood, Boston, MA (US); Patricia Murtha-Riel, North Billerica, MA (US); Gene W. Lee, Chelmsford, MA (US); Mark Leonard, Manchester, NH (US); Christopher Riel, North Billerica, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/868,373

(22) Filed: Jun. 14, 2004

Related U.S. Application Data
(60) Provisional application No. 60/477,548, filed on Jun. 11, 2003.

(51) Int. Cl. C12P 21/02; C07H 21/04; C07K 14/54

(52) U.S. Cl. 435/69.5; 435/320.1; 435/325; 530/351; 536/23.5

(57) ABSTRACT

Disclosed are methods of producing a cytokine antagonist polypeptide by co-expressing the cytokine antagonist polypeptide with a nucleic acid encoding a complexing polypeptide for the cytokine antagonist polypeptide.

### RELATED APPLICATIONS

This application claims priority to U.S. Ser. No. 60/477,548, filed Jun. 11, 2003. The contents of this application are incorporated herein by reference in their entirety.

### FIELD OF THE INVENTION

The invention relates generally to polypeptides and more specifically to cytokine antagonist polypeptides, and to methods of producing cytokine antagonist polypeptides.

### BACKGROUND OF THE INVENTION

Cytokines are polypeptides secreted by cells of the immune system and exert regulatory effects on the cells of the immune system. They have been reported to play a major role in the pathogenesis of numerous diseases, including allergic rhinitis, atopic dermatitis, allergic asthma, some parasitic infections, and cancer.

The cellular responses to cytokines are mediated through receptors found on the surfaces of responsive cells. The cytokine receptors may include intracellular, transmembrane, and extracellular components. The extracellular portion of some cytokine receptor polypeptides can be expressed in a soluble form. When added to a population of cells known to be responsive to the cognate cytokine, soluble cytokine receptor polypeptides can inhibit the function of the cytokine. For example, a polypeptide that includes the extracellular portion of the IL-13 receptor has been reported to inhibit the function of IL-13 function in vitro and in vivo.

The expression level of soluble cytokine antagonists, including inhibitors based on the extracellular portions of the IL-13 receptor polypeptide, in cell culture, however, is low. This can limit the commercial feasibility of manufacturing cytokine antagonist. Thus, there is a need for an effective method of producing a high level of a soluble cytokine antagonist from cell culture.

### SUMMARY OF THE INVENTION

The invention is based in part on the discovery of an improved method for producing an IL-13 antagonist polypeptide. The IL-13 antagonist polypeptide produced in the method is recovered in high yields and in a stable form. The method additionally results in production of a high proportion of the IL-13 antagonist polypeptide in a dimeric form, which is the most active form of the antagonist polypeptide.

The invention also provides for a pharmaceutical composition that includes the cytokine antagonist polypeptide of this method as well as a method of reducing the level of a cytokine, e.g., IL-13 in a patient that includes administering to the patient a therapeutically effective amount of this pharmaceutical composition.

In one aspect the invention provides a method of producing an IL-13 antagonist polypeptide. In the method, a culture medium is provided that includes a host cell. The host cell expresses a nucleic acid encoding the IL-13 antagonist polypeptide and the host cell expresses a nucleic acid encoding a complexing polypeptide for the IL-13 antagonist polypeptide. The host cell is cultured under conditions allowing for expression of the IL-13 antagonist polypeptide and the complexing polypeptide. The IL-13 antagonist polypeptide is recovered from the culture medium, thereby producing the IL-13 antagonist polypeptide.

Examples of suitable complexing polypeptides include IL-13 (including an IL-13 polypeptide with the amino acid sequence of a human IL-13 polypeptide), an IL-13 receptor binding fragment of an IL-13 polypeptide, an antibody to an IL-13 receptor polypeptide, and IL-6 (including an IL-6 polypeptide with the amino acid sequence of a human IL-6 polypeptide).

In some embodiments, the nucleic acid encoding the IL-13 antagonist polypeptide is a nucleic acid endogenous with respect to the host cell.

In some embodiments, the nucleic acid encoding the complexing polypeptide is an exogenous nucleic acid.

The method optionally includes introducing the exogenous nucleic acid into the host cell.

In some embodiments, more antagonist polypeptide is recovered when the IL-13 antagonist polypeptide is co-expressed with the complexing polypeptide than when the IL-13 antagonist polypeptide is expressed in the absence of the complexing polypeptide.

In some embodiments, the host cell is cultured at a temperature of from about 29° C. to about 39° C. when expressing the nucleic acid encoding the IL-13 antagonist polypeptide and the complexing polypeptide. For example the temperature can be about, e.g., 30° C., 32° C., 34° C., 36° C., or 37° C., or 38° C.

The host cell can be, e.g., a stably transfected cell (such as a stably transfected Chinese Hamster Ovary (CHO) cell). Alternatively, the host cell can be a transiently transfected cell (such as a transiently transfected COS cell).

In some embodiments, the IL-13 antagonist polypeptide includes an extracellular moiety of an IL-13 receptor polypeptide fused to at least a portion of an immunoglobulin polypeptide. Examples of an IL-13 receptor polypeptide include an IL-13Rα1, IL-13Rα2, or IL-4 receptor polypeptide chain.

In some embodiments, the IL-13 antagonist polypeptide includes an Fc region of an immunoglobulin γ1 polypeptide.

An example of an IL-13 antagonist polypeptide is IL-13 Rα.2Fc.

In some embodiments, aggregation of the expressed IL-13 antagonist polypeptide is reduced relative to aggregation of the IL-13 antagonist polypeptide expressed in a host cell not expressing the nucleic acid encoding the complexing polypeptide for the IL-13 polypeptide. For example, in various embodiments, aggregation is reduced at least about 10%, 30%, 50%, 70%, 80%, 90% or more relative to aggregation of the IL-13 antagonist polypeptide expressed in a host cell not expressing the nucleic acid encoding the complexing polypeptide for the IL-13 polypeptide.

In a further aspect, the invention provides a method of producing an IL-13 Rα2.Fc polypeptide by providing a culture medium that includes a cell, wherein the cell expresses a nucleic acid encoding IL-13 Rα2.Fc polypeptide and a nucleic acid encoding a complexing polypeptide for the IL-13 Rα2.Fc polypeptide. The cell is cultured under conditions allowing for expression of the IL-13 Rα2.Fc polypeptide and the complexing polypeptide; and the IL-13 Rα2.Fc polypeptide is recovered from the culture medium, thereby producing the IL-13 Rα2.Fc polypeptide.

Also within the invention is a method of producing an IL-13 Rα2.Fc polypeptide by providing a culture medium comprising a cell that expresses a nucleic acid encoding the IL-13 Rα2.Fc polypeptide and a nucleic acid encoding an IL-13 polypeptide. The cell is cultured under conditions allowing for expression of the IL-13 Rα2.Fc polypeptide and the IL-13 polypeptide. The IL-13 Rα2.Fc polypeptide is recovered from the culture medium, thereby producing the IL-13 Rα2.Fc polypeptide.

In some embodiments, more IL-13 Rα2.Fc polypeptide is recovered when the IL-13 Rα2.Fc polypeptide is co-expressed with IL-13 than when the IL-13 Rα2.Fc polypeptide is expressed in the absence of IL-13.

In a further aspect, the invention provides an IL-13 antagonist polypeptide (e.g., an IL-13 Rα2.Fc polypeptide) produced by the methods described herein and a pharmaceutically acceptable carrier.

In a still further aspect, the invention provides a purified preparation of a soluble IL-13 antagonist polypeptide, wherein at least 40% of the polypeptide is present as a monomer or dimer following incubation for at least one week at 4° C. In some embodiments, at least 50%, 60%, 70%, 80%, 90%, or 95% of the polypeptide is present as a monomer or dimer.

Also within the invention is method of reducing the level of a cytokine in a patient comprising administering to the patient a therapeutically effective amount of a composition that includes a cytokine polypeptide antagonist polypeptide (including an IL-13 antagonist polypeptide) described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1A is an autoradiogram showing ³⁵S-labeled polypeptides from COS cell lines.

FIG. 1B is an autoradiogram showing ³⁵S-labeled polypeptides from COS cell lines prepared by Protein A precipitation.

FIG. 2 is a schematic diagram depicting the circular map of IL-13 expression plasmid pTMNhIL13H6EK.

FIG. 3 is a graph showing the level of IL-13Rα2.Fc fusion polypeptide production of select clones bearing the pTMNhIL13H6EK plasmid.

FIG. 4A is a graph showing the effect of temperature on the time-dependent production of IL-13Rα2.Fc fusion polypeptide in 6fd3 cell line and 31b5 cell line.

FIG. 4B is a histogram showing the effect of temperature on the time-dependent production of sIL-13Rα2.Fc fusion polypeptide in the 6fd3 cell line, which expressed sIL-13R, and the 31b5 cell line, which co-expressed sIL-13R and IL-13. For each cell line and temperature, production of sIL-13Rα2.Fc fusion polypeptide, if detected, is shown at day 3, day 5, day 10, and day 14. No production at day 14 was detected at 37° C. for either 6fd3 or 31b5 cells.

FIG. 5A is a schematic representation comparing the elution profiles of IL-13Rα2.Fc fusion polypeptide molecular aggregates purified by SEC-HPLC.

FIG. 5B is a histogram showing the effect of time and temperature on the relative amounts of the major IL-13Rα2.Fc fusion polypeptide species produced by 6fd3 parental cell line and the IL-13 co-expressing 31b5 cell line. For each cell line, day and temperature, the level of the HMW2 form is presented as the first histogram, followed by a histogram showing the level of the HMW1 form. The level of the dimer form is shown as a circle for each cell line at the indicated day and temperature.

FIG. 6A is a graphic representation of the effect of 6 day storage at 4° C. on the relative distribution of major IL-13Rα2.Fc fusion polypeptide species in a preparation of Protein A-purified IL-13Rα2.Fc fusion polypeptide from 6fd3 parental cell line.

FIG. 6B is a graphic representation of the effect of 6 day storage at 4° C. on the relative distribution of major IL-13Rα2.Fc fusion polypeptide species in a preparation of Protein A-purified IL-13Rα2.Fc fusion polypeptide from the IL-13 co-expressing 37A4 cell line.

FIG. 7 is a SDS-PAGE gel showing the composition of Protein A purified preparations from 6df3 parental cell line and IL-13 co-expressing 37A4 cell line.

FIG. 8A is a histogram showing the relative amounts of HMW1, HMW2, and dimer human s13Rα2.Fc forms in Day 9 conditioned media following coexpression at 37° C. or 31° C. in the presence of no IL-13, wild-type human IL-13, R127D human IL-13, and R127P human IL-13. For each data set, the order of histograms represents the amount of (left to right) HMW1 form, HMW2 form, and dimer form.

FIG. 8B is a graphical representation showing IL-13 levels (expressed as a percentage normalized to IL-13 levels detected following solubilization with SDS) detected at increasing concentrations of MgCl₂ following expression of human s13Rα2.Fc in the presence of wild-type human IL-13, R127D human IL-13, or R127P human IL-13.

### DETAILED DESCRIPTION OF THE INVENTION

Cytokine antagonist polypeptides are produced by co-expressing a nucleic acid encoding the antagonist polypeptide along with a nucleic acid encoding a polypeptide, known as a complexing polypeptide, that complexes with the cytokine antagonist polypeptide. Co-expression increases the yield of cytokine antagonist polypeptide compared to production of the cytokine antagonist polypeptide in the absence of the complexing polypeptide. In addition, co-expression reduces the amount of high molecular weight forms of the cytokine antagonist polypeptide present in cytokine antagonist polypeptide preparations relative to the amount of high molecular weight forms observed when the cytokine antagonist polypeptide is expressed in the absence of the complexing polypeptide.

Cytokine Antagonist Polypeptides

The term “cytokine antagonist polypeptide,” as used herein, refers to any polypeptide that inhibits one or more biological activities of its cognate cytokine. Thus, a cytokine antagonist polypeptide can include a polypeptide that inhibits the activity of the corresponding cytokine. The activities inhibited can include: (1) the ability to bind a cytokine or a fragment thereof (e.g., a biologically active fragment thereof); and/or (2) the ability to interact with the second non-cytokine-binding chain of a cytokine receptor to produce a signal characteristic of the binding of cytokine to a cytokine receptor. In some embodiments, the cytokine antagonist contains an extracellular moiety of a cytokine receptor. The cytokine antagonist can also be a cytokine-binding immunoglobulin polypeptide, e.g., polyclonal antibody, monoclonal antibody, or fragment thereof.

In general, any cytokine antagonist polypeptide for which a nucleic acid sequence is known and for which a cognate ligand is known can be used. One suitable cytokine antagonist polypeptide is an IL-13 receptor fusion polypeptide, which can include a portion of an IL-13 receptor polypeptide (such as the extracellular portion) fused to a non-IL-13 receptor polypeptide, e.g., an immunoglobulin fragment. The IL-13 receptor-derived portion can be derived from an IL-13Rα1 or IL-13Rα2 receptor chain. The IL-13 receptor moiety can in addition be derived from to the amino acid sequence of any mammalian IL-13 receptor polypeptide chain, including human and rodent (such as rat or mouse).

Murine and Human Cytokine IL-13 Receptor Antagonist Polypeptide Sequences

A murine IL-13Rα1 nucleic acid sequence and its encoded polypeptide sequence of 424 amino acids is provided below as SEQ ID NO:1 and SEQ ID NO:2, respectively. These sequences are described in Hilton et al., Proc. Natl. Acad. Sci. USA, 93:497-501, 1996.

| | |
|---|---|
| TGAAAAGATAGAATAAATGGCCTCGTGCCGAATTCGG | (SEQ ID NO:1) |
| CACGAGCCGAGGCGAGGGCCTGCATGGCGCGGCCAGC | |
| GCTGCTGGGCGAGCTGTTGGTGCTGCTACTGTGGACC | |
| GCCACCGTGGGCCAAGTTGCCGCGGCCACAGAAGTTC | |
| AGCCACCTGTGACGAATTTGAGCGTCTCTGTCGAAAA | |
| TCTCTGCACGATAATATGGACGTGGAGTCCTCCTGAA | |
| GGAGCCAGTCCAAATTGCACTCTCAGATATTTTAGTC | |
| ACTTTGATGACCAACAGGATAAGAAAATTGCTCCAGA | |
| AACTCATCGTAAAGAGGAATTACCCCTGGATGAGAAA | |
| ATCTGTCTGCAGGTGGGCTCTCAGTGTAGTGCCAATG | |
| AAAGTGAGAAGCCTAGCCCTTTGGTGAAAAAGTGCAT | |
| CTCACCCCCTGAAGGTGATCCTGAGTCCGCTGTGACT | |
| GAGCTCAAGTGCATTTGGCATAACCTGAGCTATATGA | |
| AGTGTTCCTGGCTCCCTGGAAGGAATACAAGCCCTGA | |
| CACACACTATACTCTGTACTATTGGTACAGCAGCCTG | |
| GAGAAAAGTCGTCAATGTGAAAACATCTATAGAGAAG | |
| GTCAACACATTGCTTGTTCCTTTAAATTGACTAAAGT | |
| GGAACCTAGTTTTGAACATCAGAACGTTCAAATAATG | |
| GTCAAGGATAATGCTGGGAAAATTAGGCCATCCTGCA | |
| AAATAGTGTCTTTAACTTCCTATGTGAAACCTGATCC | |
| TCCACATATTAAACATCTTCTCCTCAAAAATGGTGCC | |
| TTATTAGTGCAGTGGAAGAATCCACAAAATTTTAGAA | |
| GCAGATGCTTAACTTATGAAGTGGAGGTCAATAATAC | |
| TCAAACCGACCGACATAATATTTTAGAGGTTGAAGAG | |
| GACAAATGCCAGAATTCCGAATCTGATAGAAACATGG | |
| AGGGTACAAGTTGTTTCCAACTCCCTGGTGTTCTTGC | |
| CGACGCTGTCTACACAGTCAGAGTAAGAGTCAAAACA | |
| AACAAGTTATGCTTTGATGACAACAAACTGTGGAGTG | |
| ATTGGAGTGAAGCACAGAGTATAGGTAAGGAGCAAAA | |
| CTCCACCTTCTACACCACCATGTTACTCACCATTCCA | |
| GTCTTTGTCGCAGTGGCAGTCATAATCCTCCTTTTTT | |
| ACCTGAAAAGGCTTAAGATCATTATATTTCCTCCAAT | |
| TCCTGATCCTGGCAAGATTTTTAAAGAAATGTTTGGA | |
| GACCAGAATGATGATACCCTGCACTGGAAGAAGTATG | |
| ACATCTATGAGAAACAATCCAAAGAAGAAACGGATTC | |
| TGTAGTGCTGATAGAAAACCTGAAGAAAGCAGCTCCT | |
| TGATGGGGAGAAGTGATTTCTTTCTTGCCTTCAATGT | |
| GACCCTGTGAAGATTTATTGCATTCTCCATTTGTTAT | |
| CTGGGGGACTTGTTAAATAGAAACTGAAACTACTCTT | |
| GAAAAGCAGGCAGCTCCTAAGAGCCACAGGTCTTGAT | |
| GTGACTTTTGCATTGAAAACCCAAACCCAAAGGAGCT | |
| CCTTCCAAGAAAAGCAAGAGTTCTTCTCGTTCCTTGT | |
| TCCAATCCCTAAAAGCAGATGTTTTGCCAAATCCCCA | |
| AACTAGAGGACAAAGACAAGGGGACAATGACCATCAA | |
| TTCATCTAATCAGGAATTGTGATGGCTTCCTAAGGAA | |
| TCTCTGCTTGCTCTG | |
| MARPALLGELLVLLLWTATVGQVAAATEVQPPVTNLS | (SEQ ID NO:2) |
| VSVENLCTIIWTWSPPEGASPNCTLRYFSHFDDQQDK | |
| KIAPETHRKEELPLDEKICLQVGSQCSANESEKPSPL | |
| VKKCISPPEGDPESAVTELKCIWHNLSYMKCSWLPGR | |
| NTSPDTHYTLYYWYSSLEKSRQCENIYREGQHIACSF | |
| KLTKVEPSFEHQNVQIMVKDNAGKIRPSCKIVSLTSY | |
| VKPDPPHIKHLLLKNGALLVQWKNPQNFRSRCLTYEV | |
| EVNNTQTDRHNILEVEEDKCQNSESDRNMEGTSCFQL | |
| PGVLADAVYTVRVRVKTNKLCFDDNKLWSDWSEAQSI | |
| GKEQNSTFYTTMLLTIPVFVAVAVIILLFYLKRLKII | |
| IFPPIPDPGKIFKEMFGDQNDDTLHWKKYDIYEKQSK | |
| EETDSVVLIENLKKAAP | |

A nucleic acid sequence encoding a murine IL-13Rα2 polypeptide sequence, and the encoded sequence, are presented below as SEQ ID NO:3 and SEQ ID NO:4, respectively. The encoded polypeptide has a length of 383 amino acids. Amino acids 1-332 of SEQ ID NO:4 correspond to the extracellular domain of murine IL13Rα2 polypeptide. Sequences encoding IL-13Rα2 are also discussed in Donaldson et al., J. Immunol., 161:2317-24, 1998.

| | |
|---|---|
| GGCACGAGGGAGAGGAGGAGGGAAAGATAGAAAGAGA | (SEQ ID NO:3) |
| GAGAGAAAGATTGCTTGCTACCCCTGAACAGTGACCT | |
| CTCTCAAGACAGTGCTTTGCTCTTCACGTATAAGGAA | |
| GGAAAACAGTAGAGATTCAATTTAGTGTCTAATGTGG | |
| AAAGGAGGACAAAGAGGTCTTGTGATAACTGCCTGTG | |
| ATAATACATTTCTTGAGAAACCATATTATTGAGTAGA | |
| GCTTTCAGCACACTAAATCCTGGAGAAATGGCTTTTG | |
| TGCATATCAGATGCTTGTGTTTCATTCTTCTTTGTAC | |
| AATAACTGGCTATTCTTTGGAGATAAAAGTTAATCCT | |
| CCTCAGGATTTTGAAATATTGGATCCTGGATTACTTG | |
| GTTATCTCTATTTGCAATGGAAACCTCCTGTGGTTAT | |
| AGAAAAATTTAAGGGCTGTACACTAGAATATGAGTTA | |
| AAATACCGAAATGTTGATAGCGACAGCTGGAAGACTA | |
| TAATTACTAGGAATCTAATTTACAAGGATGGGTTTGA | |
| TCTTAATAAAGGCATTGAAGGAAAGATACGTACGCAT | |
| TTGTCAGAGCATTGTACAAATGGATCAGAAGTACAAA | |
| GTCCATGGATAGAAGCTTCTTATGGGATATCAGATGA | |
| AGGAAGTTTGGAAACTAAAATTCAGGACATGAAGTGT | |
| ATATATTATAACTGGCAGTATTTGGTCTGCTCTTGGA | |
| AACCTGGCAAGACAGTATATTCTGATACCAACTATAC | |
| CATGTTTTTCTGGTATGAGGGCTTGGATCATGCCTTA | |
| CAGTGTGCTGATTACCTCCAGCATGATGAAAAAAATG | |
| TTGGATGCAAACTGTCCAACTTGGACTCATCAGACTA | |
| TAAAGATTTTTTTATCTGTGTTAATGGATCTTCAAAG | |
| TTGGAACCCATCAGATCCAGCTATACAGTTTTTCAAC | |
| TTCAAAATATAGTTAAACCATTGCCACCAGAATTCCT | |
| TCATATTAGTGTGGAGAATTCCATTGATATTAGAATG | |
| AAATGGAGCACACCTGGAGGACCCATTCCACCAAGGT | |
| GTTACACTTATGAAATTGTGATCCGAGAAGACGATAT | |
| TTCCTGGGAGTCTGCCACAGACAAAAACGATATGAAG | |
| TTGAAGAGGAGAGCAAATGAAAGTGAAGACCTATGCT | |
| TTTTTGTAAGATGTAAGGTCAATATATATTGTGCAGA | |
| TGATGGAATTTGGAGCGAATGGAGTGAAGAGGAATGT | |
| TGGGAAGGTTACACAGGGCCAGACTCAAAGATTATTT | |
| TCATAGTACCAGTTTGTCTTTTCTTTATATTCCTTTT | |
| GTTACTTCTTTGCCTTATTGTGGAGAAGGAAGAACCT | |
| GAACCCACATTGAGCCTCCATGTGGATCTGAACAAAG | |
| AAGTGTGTGCTTATGAAGATACCCTCTGTTAAACCAC | |
| CAATTTCTTGACATAGAGCCAGCCAGCAGGAGTCATA | |
| TTAAACTCAATTTCTCTTAAAATTTCGAATACATCTT | |
| CTTGAAAATCAGTGTTTGTCCTAATAGTGTTGGGTTT | |
| TTGACTAAAGTGCTGGATATATATCTCCAAAAAAAAA | |
| AAAAAAAAAAAAA | |
| MAFVHIRCLCFILLCTITGYSLEIKVNPPQDFEILDP | (SEQ ID NO:4) |
| GLLGYLYLQWKPPVVIEKFKGCTLEYELKYRNVDSDS | |
| WKTIITRNLIYKDGFDLNKGIEGKIRTHLSEHCTNGS | |
| EVQSPWIEASYGISDEGSLETKIQDMKCIYYNWQYLV | |
| CSWKPGKTVYSDTNYTMFFWYEGLDHALQCADYLQHD | |
| EKNVGCKLSNLDSSDYKDFFICVNGSSKLEPIRSSYT | |
| VFQLQNIVKPLPPEFLHISVENSIDIRMKWSTPGGPI | |
| PPRCYTYEIVIREDDISWESATDKNDMKLKRRANESE | |
| DLCFFVRCKVNIYCADDGIWSEWSEEECWEGYTGPDS | |
| KIIFIVPVCLFFIFLLLLLCLIVEKEEPEPTLSLHVD | |
| LNKEVCAYEDTLC | |

A nucleic acid sequence encoding a human IL-13Rα2 polypeptide sequence, and the encoded sequence, are presented below as SEQ ID NO:5 and SEQ ID NO:6, respectively. The encoded polypeptide has a length of 380 amino acids. A nucleic acid sequence encoding a human IL-13Rα2 polypeptide chain is shown below and is also found in Genbank Acc. No. U70981.1, as well as Caput et al., J. Biol. Chem. 271:16921-26, 1996; Zhang et al., J. Biol. Chem. 272:9474-78, 1997; and Guo et al., Genomics 42:141-45, 1997. The open reading frame encoding the IL-13Rα2 polypeptide begins with the highlighted ATG codon and ends with the highlighted TGA codon. The first 27 amino acids of the encoded polypeptide correspond to an amino terminal signal sequence. A suitable polypeptide that includes the extracellular portion of the IL-13 receptor includes the 313 amino acid polypeptide fragment that includes amino acids 28-340 (shown in bold).

| | |
|---|---|
| CGGATGAAGGCTATTTGAAGTCGCCATAACCTGGTCA | (SEQ ID NO:5) |
| GAAGTGTGCCTGTCGGCGGGGAGAGAGGCAATATCAA | |
| GGTTTTAAATCTCGGAGAAATGGCTTTCGTTTGCTTG | |
| GCTATCGGATGCTTATATACCTTTCTGATAAGCACAA | |
| CATTTGGCTGTACTTCATCTTCAGACACCGAGATAAA | |
| AGTTAACCCTCCTCAGGATTTTGAGATAGTGGATCCC | |
| GGATACTTAGGTTATCTCTATTTGCAATGGCAACCCC | |
| CACTGTCTCTGGATCATTTTAAGGAATGCACAGTGGA | |
| ATATGAACTAAAATACCGAAACATTGGTAGTGAAACA | |
| TGGAAGACCATCATTACTAAGAATCTACATTACAAAG | |
| ATGGGTTTGATCTTAACAAGGGCATTGAAGCGAAGAT | |
| ACACACGCTTTTACCATGGCAATGCACAAATGGATCA | |
| GAAGTTCAAAGTTCCTGGGCAGAAACTACTTATTGGA | |
| TATCACCACAAGGAATTCCAGAAACTAAAGTTCAGGA | |
| TATGGATTGCGTATATTACAATTGGCAATATTTACTC | |
| TGTTCTTGGAAACCTGGCATAGGTGTACTTCTTGATA | |
| CCAATTACAACTTGTTTTACTGGTATGAGGGCTTGGA | |
| TCATGCATTACAGTGTGTTGATTACATCAAGGCTGAT | |
| GGACAAAATATAGGATGCAGATTTCCCTATTTGGAGG | |
| CATCAGACTATAAAGATTTCTATATTTGTGTTAATGG | |
| ATCATCAGAGAACAAGCCTATCAGATCCAGTTATTTC | |
| ACTTTTCAGCTTCAAAATATAGTTAAACCTTTGCCGC | |
| CAGTCTATCTTACTTTTACTCGGGAGAGTTCATGTGA | |
| AATTAAGCTGAAATGGAGCATACCTTTGGGACCTATT | |
| CCAGCAAGGTGTTTTGATTATGAAATTGAGATCAGAG | |
| AAGATGATACTACCTTGGTGACTGCTACAGTTGAAAA | |
| TGAAACATACACCTTGAAAACAACAAATGAAACCCGA | |
| CAATTATGCTTTGTAGTAAGAAGCAAAGTGAATATTT | |
| ATTGCTCAGATGACGGAATTTGGAGTGAGTGGAGTGA | |
| TAAACAATGCTGGGAAGGTGAAGACCTATCGAAGAAA | |
| ACTTTGCTACGTTTCTGGCTACCATTTGGTTTCATCT | |
| TAATATTAGTTATATTTGTAACCGGTCTGCTTTTGCG | |
| TAAGCCAAACACCTACCCAAAAATGATTCCAGAATTT | |
| TTCTGTGATACATGAAGACTTTCCATATCAAGAGACA | |
| TGGTATTGACTCAACAGTTTCCAGTCATGGCCAAATG | |
| TTCAATATGAGTCTCAATAAACTGAATTTTTCTTGCG | |
| AAAAAAAAAAAAAAAAAAAAAAAAAAAAAAAAAAAAA | |
| AAAAAAAAAAAAA | |
| MAFVCLAIGCLYTFLISTTFGCTSSSDTEIKVNPPQD | (SEQ ID NO:6) |
| FEIVDPGYLGYLYLQWQPPLSLDHFKECTVEYELKYR | |
| NIGSETWKTIITKNLHYKDGFDLNKGIEAKIHTLLPW | |
| QCTNGSEVQSSWAETTYWISPQGIPETKVQDMDCVYY | |
| NWQYLLCSWKPGIGVLLDTNYNLFYWYEGLDHALQCV | |
| DYIKADGQNIGCRFPYLEASDYKDFYICVNGSSENKP | |
| IRSSYFTFQLQNIVKPLPPVYLTFTRESSCEIKLKWS | |
| IPLGPIPARCFDYEIEIREDDTTLVTATVENETYTLK | |
| TTNETRQLCFVVRSKVNIYCSDDGIWSEWSDKQCWEG | |
| EDLSKKTLLRFWLPFGFILILVIFVTGLLLRKPNTYP | |
| KMIPEFFCDT. | |

Non-Cytokine-Receptor Polypeptides Present in the Cytokine Antagonist Polypeptide

The cytokine antagonist polypeptide can include an immunoglobulin moiety (such as an Fc region of an immunoglobulin γ-1 polypeptide; Caput et al., J. Biol. Chem. 271:16921-29, 1996; Donaldson et al., J. Immunol. 161:2317-24, 1998). Other suitable non-IL-13-receptor polypeptide sequences include, e.g., GST, Lex-A, or MBP moieties. The fusion polypeptide may in addition contain modifications (such as pegylated moieties) that enhance its stability.

The nucleotide sequence and encoded 330 amino acid sequence of human Ig γ-1 chain constant region amino acid sequence are shown below as SEQ ID NO:7 and SEQ ID NO:8, respectively. They are also described in Ellison et al., Nucleic Acids Res., 10:4071-9, 1982:

| | |
|---|---|
| AGCTTTCTGGGGCAGGCCAGGCCTGACCTTGGCTTTG | (SEQ ID NO:7) |
| GGGCAGGGAGGGGGCTAAGGTGAGGCAGGTGGCGCCA | |
| GCCAGGTGCACACCCAATGCCCATGAGCCCAGACACT | |
| GGACGCTGAACCTCGCGGACAGTTAAGAACCCAGGGG | |
| CCTCTGCGCCCTGGGCCCAGCTCTGTCCCACACCGCG | |
| GTCACATGGCACCACCTCTCTTGCAGCCTCCACCAAG | |
| GGCCCATCGGTCTTCCCCCTGGCACCCTCCTCCAAGA | |
| GCACCTCTGGGGGCACAGCGGCCCTGGGCTGCCTGGT | |
| CAAGGACTACTTCCCCGAACCGGTGACGGTGTCGTGG | |
| AACTCAGGCGCCCTGACCAGCGGCGTGCACACCTTCC | |
| CGGCTGTCCTACAGTCCTCAGGACTCTACTCCCTCAG | |
| CAGCGTGGTGACCGTGCCCTCCAGCAGCTTGGGCACC | |
| CAGACCTACATCTGCAACGTGAATCACAAGCCCAGCA | |
| ACACCAAGGTGGACAAGAAAGTTGGTGAGAGGCCAGC | |
| ACAGGGAGGGAGGGTGTCTGCTGGAAGCCAGGCTCAG | |
| CGCTCCTGCCTGGACGCATCCCGGCTATGCAGCCCCA | |
| GTCCAGGGCAGCAAGGCAGGCCCCGTCTGCCTCTTCA | |
| CCCGGAGGCCTCTGCCCGCCCCACTCATGCTCAGGGA | |
| GAGGGTCTTCTGGCTTTTTCCCCAGGCTCTGGGCAGG | |
| CACAGGCTAGGTGCCCCTAACCCAGGCCCTGCACACA | |
| AAGGGGCAGGTGCTGGGCTCAGACCTGCCAAGAGCCA | |
| TATCCGGGAGGACCCTGCCCCTGACCTAAGCCCACCC | |
| CAAAGGCCAAACTCTCCACTCCCTCAGCTCGGACACC | |
| TTCTCTCCTCCCAGATTCCAGTAACTCCCAATCTTCT | |
| CTCTGCAGAGCCCAAATCTTGTGACAAAACTCACACA | |
| TGCCCACCGTGCCCAGGTAAGCCAGCCCAGGCCTCGC | |
| CCTCCAGCTCAAGGCGGGACAGGTGCCCTAGAGTAGC | |
| CTGCATCCAGGGACAGGCCCCAGCCGGGTGCTGACAC | |
| GTCCACCTCCATCTCTTCCTCAGCACCTGAACTCCTG | |
| GGGGGACCGTCAGTCTTCCTCTTCCCCCCAAAACCCA | |
| AGGACACCCTCATGATCTCCCGGACCCCTGAGGTCAC | |
| ATGCGTGGTGGTGGACGTGAGCCACGAAGACCCTGAG | |
| GTCAAGTTCAACTGGTACGTGGACGGCGTGGAGGTGC | |
| ATAATGCCAAGACAAAGCCGCGGGAGGAGCAGTACAA | |
| CAGCACGTACCGTGTGGTCAGCGTCCTCACCGTCCTG | |
| CACCAGGACTGGCTGAATGGCAAGGAGTACAAGTGCA | |
| AGGTCTCCAACAAAGCCCTCCCAGCCCCCATCGAGAA | |
| AACCATCTCCAAAGCCAAAGGTGGGACCCGTGGGGTG | |
| CGAGGGCCACATGGACAGAGGCCGGCTCGGCCCACCC | |
| TCTGCCCTGAGAGTGACCGCTGTACCAACCTCTGTCC | |
| CTACAGGGCAGCCCCGAGAACCACAGGTGTACACCCT | |
| GCCCCCATCCCGGGATGAGCTGACCAAGAACCAGGTC | |
| AGCCTGACCTGCCTGGTCAAAGGCTTCTATCCCAGCG | |
| ACATCGCCGTGGAGTGGGAGAGCAATGGGCAGCCGGA | |
| GAACAACTACAAGACCACGCCTCCCGTGCTGGACTCC | |
| GACGGCTCCTTCTTCCTCTACAGCAAGCTCACCGTGG | |
| ACAAGAGCAGGTGGCAGCAGGGGAACGTCTTCTCATG | |
| CTCCGTGATGCATGAGGCTCTGCACAACCACTACACG | |
| CAGAAGAGCCTCTCCCTGTCTCCGGGTAAATGAGTGC | |
| GACGGCCGGCAAGCCCCCGCTCCCCGGGCTCTCGCGG | |
| TCGCACGAGGATGCTTGGCACGTACCCCCTGTACATA | |
| CTTCCCGGGCGCCCAGCATGGAAATAAAGCACCCAGC | |
| GCTGCCCTGGGCCCCTGCGAGACTGTGATGGTTCTTT | |
| CCACGGGTCAGGCCGAGTCTGAGGCCTGAGTGGCATG | |
| AGGGAGGCAGAGCGGGTCCCACTGTCCCCACACTGGC | |
| CCAGGCTGTGCAGGTGTGCCTGGGCCCCCTAGGGTGG | |
| GGCTCAGCCAGGGGCTGCCCTCGGCAGGGTGGGGGAT | |
| TTGCCAGCGTGGCCCTCCCTCCAGCAGCACCTGCCCT | |
| GGGCTGGGCCACGGGAAGCCCTAGGAGCCCCTGGGGA | |
| CAGACACACAGCCCCTGCCTCTGTAGGAGACTGTCCT | |
| GTTCTGTGAGCGCCCCTGTCCTCCCGACCTCCATGCC | |
| CACTCGGGGGCATGCCTAGTCCATGTGCGTAGGGACA | |
| GGCCCTCCCTCACCCATCTACCCCCACGGCACTAACC | |
| CCTGGCTGCCCTGCCCAGCCTCGCACCCGCATGGGGA | |
| CACAACCGACTCCGGGGACATGCACTCTCGGGCCCTG | |
| TGGAGGGACTGGTGCAGATGCCCACACACACACTCAG | |
| CCCAGACCCGTTCAACAAACCCCGCACTGAGGTTGGC | |
| CGGCCACACGGCCACCACACACACACGTGCACGCCTC | |
| ACACACGGAGCCTCACCCGGGCGAACTGCACAGCACC | |
| CAGACCAGAGCAAGGTCCTCGCACACGTGAACACTCC | |
| TCGGACACAGGCCCCCACGAGCCCCACGCGGCACCTC | |
| AAGGCCCACGAGCCTCTCGGCAGCTTCTCCACATGCT | |
| GACCTGCTCAGACAAACCCAGCCCTCCTCTCACAAGG | |
| GTGCCCCTGCAGCCGCCACACACACACAGGGGATCAC | |
| ACACCACGTCACGTCCCTGGCCCTGGCCCACTTCCCA | |
| GTGCCGCCCTTCCCTGCAGACGGATCC | |
| ASTKGPSVFPLAPSSKSTSGGTAALGCLVKDYFPEPV | (SEQ ID NO:8) |
| TVSWNSGALTSGVHTFPAVLQSSGLYSLSSVVTVPSS | |
| SLGTQTYICNVNHKPSNTKVDKKVEPKSCDKTHTCPP | |
| CPAPELLGGPSVFLFPPKPKDTLMISRTPEVTCVVVD | |
| VSHEDPEVKFNWYVDGVEVHNAKTKPREEQYNSTYRV | |
| VSVLTVLHQDWLNGKEYKCKVSNKALPAPIEKTISKA | |
| KGQPREPQVYTLPPSRDELTKNQVSLTCLVKGFYPSD | |
| IAVEWESNGQPENNYKTTPPVLDSDGSFFLYSKLTVD | |
| KSRWQQGNVFSCSVMHEALHNHYTQKSLSLSPGK | |

A cytokine antagonist polypeptide may additionally include heterologous leader sequences on its amino terminal end (such as the signal peptide sequence derived from the honeybee mellitin leader (HBL) sequence). In addition, nucleic acids encoding cytokine antagonist polypeptides can be engineered to include additional amino acids between the IL-13 receptor-derived sequence and a heterologous non-IL-13 polypeptide.

The construction and sequence of a nucleic acid encoding the IL-13 cytokine antagonist polypeptide hIL-13Rα2.Fc are shown in Example 1.

Complexing Polypeptide

A complexing polypeptide includes any polypeptide that binds to the cytokine antagonist polypeptide during co-expression of nucleic acids encoding the cytokine antagonist polypeptide and complexing polypeptide so as to facilitate expression of the cytokine antagonist polypeptide. Thus, a complexing polypeptide includes a polypeptide that, when co-expressed with a nucleic acid encoding a corresponding cytokine antagonist polypeptide, reduces the aggregation state, i.e., amount of aggregation or rate of aggregation, of cytokine antagonist polypeptide relative to the aggregation state of the cytokine antagonist in the absence of the complexing polypeptide.

Suitable complexing polypeptides include, e.g., the cognate cytokine polypeptide, or a cytokine antagonist-binding fragment of the cytokine polypeptide. When the cytokine antagonist polypeptide is derived from an IL-13 receptor polypeptide, the complexing polypeptide can be, e.g., IL-13, IL-6, or a fragment or mutant which binds to an IL-13 receptor polypeptide. The amino acid sequence of a human IL-13 polypeptide is disclosed in. GenBank Accession No. P35225 and Minty et al., Nature 362: 248-250, 1993. The sequence is also shown below:

| | |
|---|---|
| MALLLTTVIALTCLGGFASPGPVPPSTALRELIEEL | (SEQ ID NO:17) |
| VNITQNQKAPLCNGSMVWSINLTAGMYCAALESLIN | |
| VSGCSAIEKTQRMLSGFCPHKVSAGQFSSLHVRDTK | |
| IEVAQFVKDLLLHLKKLFREGRFN | |

Another suitable complexing polypeptide is an IL-13 variant polypeptide with the arginine at position 127 replaced with any of the other 19 encoded amino acids. In some embodiments, the arginine is replaced with aspartic acid, glutamic acid, or proline residue (referred to herein as R127D, R127E, and R127P variants). It has been unexpectedly found that the R127D and R127P variants are more easily separated from solubilized from the IL-13 receptor during purification than the corresponding polypeptide with arginine at position 127.

An additional suitable complexing polypeptide is an antibody that binds to the cytokine antagonist polypeptide. The antibody can be either a polyclonal antibody or a monoclonal antibody. Antibodies to the cytokine antagonist can be made using techniques known in the art. For example, an extracellular portion of a cytokine antagonist may be used to immunize animals to obtain polyclonal and monoclonal antibodies which specifically react with the cytokine antagonist protein. Such antibodies may be obtained using the entire cytokine antagonist as an immunogen, or by using fragments of cytokine antagonist, for example, a fragment of a cytokine receptor such as IL-13Rα2. Smaller fragments of cytokine antagonist may also be used to immunize animals. Methods for synthesizing such peptides are known in the art, for example, as described in Merrifield, J. Amer. Chem. Soc., 85:2149-2154, 1963.

Vectors

Nucleic acids expressing a cytokine antagonist and a complexing polypeptide for the cytokine antagonist may be provided in vectors to propagate replication of the nucleic acids in a host cell. Vectors will typically include a selectable marker that allows for detection and/or selection of the gene in a host cell. Markers can include, e.g., antibiotic resistance genes, and genes encoding enzymes that catalyze metabolic reactions.

The vector can be extrachromosomal or can direct integration of the sequences into an endogenous chromosome of the host cell. The vector can additionally include sequences that promote replication of linked sequences. An example of such a sequence is an origin of replication or autonomously replicating sequence (ARS). The nucleic acids expressing the cytokine antagonist can be present on the same nucleic acid as the nucleic acid encoding its complexing polypeptide; alternatively, the nucleic acids can be present on different nucleic acids.

Expression vectors can be used to express nucleic acids encoding the cytokine antagonist and a complexing polypeptide. The sequences are assembled in an appropriate phase with translation initiation and termination sequences. If desired, a leader sequence capable of directing secretion of translated protein into the periplasmic space or extracellular medium may be incorporated. Optionally, a heterologous sequence can encode a fusion protein including an amino terminal identification peptide imparting desired characteristics, e.g., stabilization or simplified purification of the expressed recombinant product.

Expression vectors include one or more expression control sequences that modulate transcription, RNA processing, and/or translation of cytokine antagonist and complexing polypeptide nucleic acids. Such expression control sequences are known in the art and include, e.g., a promoter, an enhancer, ribosome-binding sites, RNA splice sites, polyadenylation sites, transcriptional terminator sequences, and mRNA stabilizing sequences. Suitable enhancer and other expression control sequences are discussed in, e.g., Enhancers and Eukaryotic Gene Expression, Cold Spring Harbor Press, Cold Spring Harbor, N.Y. (1983), U.S. Pat. Nos. 5,691,198; 5,735,500; 5,747,469 and 5,436,146. Expression control sequences can include, e.g., early and late promoters from SV40, promoter sequences derived from retroviral long terminal repeats (including murine Moloney leukemia virus, mouse tumor virus, avian sarcoma viruses), adenovirus II, bovine papilloma virus, polyoma virus, CMV immediate early, HSV thymidine kinase, and mouse metallothionein-I transcription enhancer sequences. Additional promoters include those derived from a highly-expressed genes, such as glycolytic enzymes (including 3-phosphoglycerate kinase (PGK)), acidic phosphatase, or genes for heat shock proteins

Suitable vectors and promoters are known to those skilled in the art and include, e.g., pWLneo, pSV2cat, pOG44, PXTI, pSG (Stratagene), pSVK3, pBPV, pMSG, pSVL (Pharmacia), the pMT2 or pED expression vectors disclosed in Kaufman, et al., Nucleic Acids Res. 19:4485-90, 1991. pTMED or pHTOP expression vector may also be used. Expression vectors may be alternatively prepared using standard recombinant techniques (See, e.g., Sambrook, et al. Molecular Cloning: A Laboratory Manual (Cold Spring Harbor Press: New York).

If desired, the nucleic acids encoding the cytokine antagonist polypeptide and/or its complexing polypeptide may be linked to a gene whose copy number in a cell can be increased. An example of such a gene is dihydrofolate reductase.

Cells

The invention also includes cells that contain vectors carrying the nucleic acids encoding the cytokine antagonist and the complexing polypeptide. A cell may include a nucleic acid that includes both the cytokine antagonist encoding sequence and the nucleic acid sequence encoding the complexing polypeptide. Alternatively, a cell can include separate nucleic acids for the cytokine antagonist encoding sequence and the complexing polypeptide encoding sequence.

In general, any cell type can be used as long as it is capable of expressing functional cytokine antagonist and complexing polypeptide protein such that they interact in a manner that facilitates subsequent purification of the cytokine antagonist. The cell can be either a prokaryotic or a eukaryotic cell. Suitable eukaryotic cells include, e.g., a mammalian cell, an insect cell (including Sf9 cells) or a yeast cell. Suitable mammalian host cells include, for example COS-7 lines of monkey kidney fibroblasts described by Gluzman, Cell 23:175, 1981; C127 monkey COS cells; Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells, COS cells, Rat2, BaF3, 32D, FDCP-1, PC12, M1x or C2C12 cells. In some embodiments, the host cell normally does not express the cytokine antagonist and/or complexing polypeptide, or express it in low levels.

Examples of yeast strains include Saccharomyces cerevisiae, Schizosaccharomyces pombe, Kluyveromyces spp. strains, and Candida spp. Examples of bacterial strains include Escherichia coli, Bacillus subtilis, and Salmonella typhimurium.

The expressed proteins can be modified post-translationally if desired, e.g., by phosphorylation or glycosylation, to enhance the function of the proteins. Such covalent attachments may be accomplished using known chemical or enzymatic methods.

The cells can be transiently transfected or permanently transfected with nucleic acids encoding the cytokine antagonist polypeptide and its complexing polypeptide.

Expressing a Cytokine Antagonist Polypeptide in the Presence of its Complexing Polypeptide

Cytokine antagonist polypeptide is prepared by growing a culture of transformed host cells under culture conditions that allow for expression of the cytokine antagonist polypeptide and the complexing polypeptide. The resulting expressed cytokine antagonist polypeptide is then purified from the culture medium or cell extracts. The cytokine antagonist polypeptide can be isolated alone or as part of a complex of other proteins (including the complexing polypeptide).

Membrane-associated forms of cytokine antagonist polypeptide are purified by preparing a total membrane fraction from the expressing cell and extracting the membranes with a non-ionic detergent such as Triton X-100. Various methods of protein purification are well known in the art, and include those described in Deutscher, ed., Guide to Protein Purification, Methods in Enzymology, vol. 182, 1990. The resulting expressed protein may then be recovered using known purification processes, such as gel filtration and ion exchange chromatography. Alternatively, the polypeptides may be purified by immunoaffinity chromatography, as described in Donaldson et al., J. Immunol. 161:2317-24, 1998.

The cytokine antagonist polypeptide can be concentrated, e.g., using a concentrating filter, for example, an Amicon or Millipore Pellicon ultrafiltration unit. Following the concentration step, the concentration can be applied to a purification matrix such as a gel filtration medium. Alternatively, an anion exchange resin can be used to purify the cytokine antagonist polypeptide. Suitable resins include, e.g., a matrix or substrate having pendant diethylaminoethyl (DEAE) or polyethelenimine (PEI) groups. The matrices can be acrylamide, agarose, dextran, cellulose or other types commonly used in protein purification. Alternatively, a cation exchange step can be used. Suitable cation exchangers include various insoluble matrices that includes sulfopropyl (e.g., S-Sepharose columns) or carboxymethyl groups. The purification of the cytokine antagonist from culture supernatant may also include one or more column steps over such affinity resins as concanavalin A-agarose, heparintoyopearl or Cibacrom blue 3GA Sepharose; or by hydrophobic interaction chromatography using such affinity resins as phenyl ether, butyl ether, or propyl ether; or by immunoaffinity chromatography. Finally, one or more reverse phase high performance liquid chromatography (RP-HPLC) steps employing hydrophobic RP-HPLC media, e.g., silica gel having pendant methyl or other aliphatic groups can be used to further purify the cytokine antagonist polypeptide. Affinity columns including cytokine antagonist or fragments thereof or including antibodies to the cytokine antagonist as well as Protein A sepharose, e.g., to facilitate purification of fusion protein containing immunoglobulin polypeptide, can also be used in purification in accordance with known methods. Some or all of the foregoing purification steps, in various combinations or with other known methods can also be used to provide a substantially purified isolated recombinant protein. In some embodiments, the isolated cytokine antagonist is purified so that it is substantially free of other proteins with which it associates in the cell expressing the polypeptide.

The cytokine antagonist protein and/or its cognate ligand can also be expressed in a form that facilitates their subsequent purification. For example, the nucleic acid encoding the cytokine antagonist can be fused in-frame to a non-cytokine antagonist sequence such as, e.g., maltose binding protein (MBP), glutathione-S-transferase (GST), thioredoxin (TRX), a His tag, or a hemagglutinin (HA) tag. The latter tag corresponds to an epitope derived from the influenza hemagglutinin protein (Wilson, et al., Cell, 37:767 (1984)). Kits for expression and purification of such fusion proteins are commercially available from New England BioLab (Beverly, Mass.), Pharmacia (Piscataway, N.J.) and Invitrogen, respectively. The protein can alternatively also be tagged with an epitope and subsequently purified by using a specific antibody directed to the epitope. An example of this epitope is the FLAG® epitope (Kodak, New Haven, Conn.). The tagged antagonist complex can be purified from the culture medium using the appropriate tag-specific method. The cytokine antagonist can be subsequently separated from its complexing polypeptide.

The cytokine antagonist protein produced by the methods described herein can be used to treat any condition for which inhibition of the activity of the corresponding cytokine is desired. When the cytokine antagonist protein is an IL-13 antagonist, the protein can be used for treatment or modulation of various medical conditions in which IL-13 is implicated or which are effected by the activity of IL-13 (collectively “IL-13-related conditions”). IL-13-related conditions include without limitation Ig-mediated conditions and diseases, particularly IgE-mediated conditions (including without limitation allergic conditions, asthma, immune complex disease (such as, for example, lupus, nephrotic syndrome, nephritis, glomerulonephritis, thyroiditis and Grave's disease)), fibrosis (including hepatic fibrosis); immune deficiencies, specifically deficiencies in hematopoietic progenitor cells, or disorders relating thereto; cancer and other disease. Such pathological states may result from disease, exposure to radiation or drugs, and include, for example, leukopenia, bacterial and viral infections, anemia, B cell or T cell deficiencies such as immune cell or hematopoietic cell deficiency following a bone marrow transplantation. An IL-13 cytokine antagonist polypeptide produced according to the methods described herein is also useful for enhancing macrophage activation (i.e., in vaccination, treatment of mycobacterial or intracellular organisms, or parasitic infections).

The cytokine antagonist polypeptide can also be used as a pharmaceutical composition when combined with a pharmaceutically acceptable carrier. Such a composition may contain, in addition to IL-13 or inhibitor and carrier, various diluents, fillers, salts, buffers, stabilizers, FN (SEQ ID NO:17) solubilizers, and other materials well known in the art. The term “pharmaceutically acceptable” means a non-toxic material that does not interfere with the effectiveness of the biological activity of the active ingredient(s). The characteristics of the carrier will depend on the route of administration.

The pharmaceutical composition may also contain additional agents, including other cytokines, lymphokines, or other hematopoietic factors such as M-CSF, GM-CSF, IL-1, IL-2, IL-3, IL-4, IL-5, IL-6, IL-7, IL-8, IL-9, IL-10, IL-11, IL-12, IL-14, IL-15, G-CSF, stem cell factor, and erythropoietin. The pharmaceutical composition may also include anti-cytokine antibodies. The pharmaceutical composition may contain thrombolytic or anti-thrombotic factors such as plasminogen activator and Factor VIII. The pharmaceutical composition may further contain other anti-inflammatory agents. Such additional factors and/or agents may be included in the pharmaceutical composition to produce a synergistic effect with the cytokine antagonist polypeptide, or to minimize side effects caused by the cytokine antagonist polypeptide.

The pharmaceutical composition may be in the form of a liposome in which the cytokine antagonist polypeptide is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871;.U.S. Pat. No.4,501,728; U.S. Pat. No. 4,827,028; and U.S. Pat. No. 4,737,323, all of which are incorporated herein by reference.

As used herein, the term “therapeutically effective amount” means the total amount of each active component of the pharmaceutical composition or method that is sufficient to show a meaningful patient benefit, e.g., amelioration of symptoms of, healing of, or increase in rate of healing of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of the cytokine antagonist polypeptide is administered to a mammal. The cytokine antagonist polypeptide may be administered either alone or in combination with other therapies such as treatments employing cytokines, lymphokines or other hematopoietic factors. When co-administered with one or more cytokines, lymphokines or other hematopoietic factors, cytokine antagonist polypeptide may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering the cytokine antagonist polypeptide in combination with cytokine(s), lymphokine(s), other hematopoietic factor(s), thrombolytic or anti-thrombotic factors.

Administration of the cytokine antagonist polypeptide used in the pharmaceutical composition or to practice the method of the present invention can be carried out in a variety of conventional ways, such as oral ingestion, inhalation, or cutaneous, subcutaneous, or intravenous injection.

When a therapeutically effective amount of cytokine antagonist polypeptide is administered orally, the cytokine antagonist polypeptide will be provided in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% of the cytokine antagonist polypeptide, e.g., about 25 to 90% of the cytokine antagonist polypeptide. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solutions, or glycols such as ethylene glycol, propylene glycol or polyethylene glycol. When administered in liquid form, the pharmaceutical composition contains from about 0.5 to 90% by weight of the cytokine antagonist polypeptide or the cytokine antagonist polypeptide. For example, in some embodiments it contains from about 1 to 50% of the cytokine antagonist polypeptide.

When a therapeutically effective amount of the cytokine antagonist polypeptide is administered by intravenous, cutaneous or subcutaneous injection, the cytokine antagonist polypeptide inhibitor will be in the form of a pyrogen-free, parenterally acceptable aqueous solution. The preparation of such parenterally acceptable protein solutions, having due regard to pH, isotonicity, stability, and the like, is within the skill in the art. In some embodiments, a pharmaceutical composition for intravenous, cutaneous, or subcutaneous injection contains, in addition to the cytokine antagonist polypeptide inhibitor, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The amount of the cytokine antagonist polypeptide in the pharmaceutical composition will depend upon the nature and severity of the condition being treated, and on the nature of prior treatments which the patient has undergone. It is contemplated that the various pharmaceutical compositions used to practice the method of the present invention will contain about 0.1 μg to about 100 mg of the cytokine antagonist polypeptide per kg body weight.

The duration of intravenous therapy using the pharmaceutical composition of the present invention will vary, depending on the severity of the disease being treated and the condition and potential idiosyncratic response of each individual patient. It is contemplated that the duration of each application of the cytokine antagonist polypeptide will be in the range of 12 to 24 hours of continuous intravenous administration. Ultimately the attending physician will decide on the appropriate duration of intravenous therapy using the pharmaceutical composition of the present invention.

The invention will be further illustrated in the following non-limiting examples.

### EXAMPLES

### Example 1

### Preparation, Expression and Characterization of Human IL-13Rα2.Fc Expression

A recombinant soluble human IL-13Rα2 fusion protein was constructed and named hIL-13Rα2.Fc.

First, nucleic acids encoding human IL-13 receptor sequences were identified using murine IL-13 receptor sequences as probes. The identification, cloning and sequencing of the murine IL-13Rα2 has been described previously (Donaldson, et al. J. Immunol., 161:2317-24, 1998). Oligonucleotide primers derived from the murine sequence were used to isolate a partial fragment of the human homologue by polymerase chain reaction with AMPLITAQ™ polymerase (Promega). The cDNA was prepared using human testis polyA+ RNA obtained from Clontech. A 274 bp fragment was identified following amplification using the primers ATAGTTAAACCATTGCCACC (SEQ ID NO:9) and CTCCATTCGCTCCAAATTCC (SEQ ID NO: 10). The sequence of the amplified fragment was used to design additional oligonucleotides for identifying additional hIL-13Rα2 sequences from a cDNA library. The sequences of the prepared oligonucleotides were AGTCTATCTTACTTTTACTCG (SEQ ID NO:11) and CATCTGAGCAATAAATATTCAC (SEQ ID NO: 12).

After labeling with ³²P, the oligonucleotides were used to screen a human testis cDNA library (Clontech). Of over 400,000 clones screened, 22 clones were identified that hybridized to both oligonucleotide probes. DNA sequence analysis was performed on four of these clones, and all four encoded the same sequence. The full-length sequence of the hIL-13Rα2 cDNA has been deposited with GenBank (accession number U70981).

The hIL-13Rα2 cDNA is predicted to encode a receptor chain with an N-terminal extracellular domain, a short trans-membrane region, and a short C-terminal cytoplasmic tail.

A soluble hIL-13Rα2 receptor that retains its ability to bind to hIL-13 was constructed by fusing the 313 NH₂-terminal amino acids from the extracellular domain of hIL-13Rα2 to the COOH-terminal 231 amino acids of a human Ig γ-1 heavy chain, which includes the hinge-CH2-CH3 region (“hIL-13Rα2.Fc”). The sequence encoding the fusion protein (termed “L2I”) was cloned into the pED vector for evaluation in COS cell transient transfection assays and in the pHTOP vector for evaluation of expression in CHO stable cell lines.

Expression of the hIL-13Rα2.Fc polypeptide in CHO cells resulted in heterogeneous NH2-terminal signal sequence processing. The natural leader sequence was therefore replaced with a leader sequence derived from the honeybee mellitin gene, which has been shown to direct efficient processing of the signal peptide (Tessier et al., Gene 98:177-83,1991). The molecule containing the honeybee leader sequence, the extracellular domain of hIL-13Rα2 and the COOH-terminus of human Ig γ-1 heavy chain was processed by the CHO cells to yield soluble hIL-13Rα2.Fc polypeptide.

The hIL-13Rα2.Fc construct was subcloned into the expression vector pTMED to permit high level gene expression in CHO cells and to allow for the selection and amplification of stable cell lines following transfection. The pHTOP-L2I plasmid was digested with the restriction enzyme NotI, blunt ended by incubation with Klenow enzyme, then digested with the restriction enzyme ApaI to liberate a 1836 bp fragment containing the entire hIL-13Rα2.Fc coding region and part of the EMCV internal ribosome entry sequence. The fragment was ligated to the pTMED plasmid previously digested with XbaI, blunt ended with Klenow, and digested with ApaI to generate the expression plasmid pTMED-L2I. DNA sequencing of the entire plasmid confirmed that the intended construct was made. The complete DNA sequence of the pTMED-L2I expression plasmid and the predicted translation product of the hIL-13Rα2.Fc gene are shown above.

The hIL-13Rα2.Fc gene was transcribed as part of a bicistronic message, with the hIL-13Rα2.Fc gene placed upstream of an encephalomyocarditis (EMC) virus internal ribosome entry site (IRES) and the selectable/amplifiable marker gene dihydrofolate reductase (DHFR). The DHFR gene conferred the ability of transfected CHO dhfr⁻ cells to grow in the absence of exogenously-added nucleosides. Transcription of the bicistronic message was driven by murine cytomegalovirus (CMV) enhancer and promoter sequences upstream of the hIL-13Rα2.Fc gene. The adenovirus tripartite leader sequence and a hybrid intervening sequence follow the CMV enhancer/promoter sequences and promote efficient translation of the bicistronic message. A signal peptide sequence derived from the honeybee mellitin gene was located immediately upstream of the hIL-13Rα2.Fc coding region.

Northern and Western blot analyses confirmed that the expression plasmid generated message and protein of the predicted size, i.e., ˜3800 nucleotides, assuming a poly(A) tail of ˜200 nucleotides, and functional evaluations performed with purified hIL-13Rα2.Fc protein demonstrated that this protein specifically binds hIL-13 and prevents the interaction of hIL-13 with cellular receptors in vitro. Southern blot analysis and genomic DNA sequencing confirmed the insertion of the expression plasmid into the host cell genome. Together, these results demonstrated that the production cell line expresses the expected hIL-13Rα2.Fc protein.

The nucleotide sequence of the pTMED-L2I expression plasmid is shown below. Nucleotide sequences corresponding to the hIL-13Rα2.Fc and DHFR coding regions are underlined. The encoded amino acid sequence of hIL-13Rα2.Fc is shown below each codon. The signal peptide sequence derived from the honeybee mellitin leader (HBL) is underlined. The amino acid sequences corresponding to the extracellular region of hIL-13Rα2 are shown in bold.

Nucleotide Sequence of pTMED-L2I Expression Plasmid and Amino Acid Sequence
of hIL-13Rα2.Fc+HZ,1/49

| | |
|---|---|
| 1 | |
| CATATGCGGTGTGAAATACCGCACAGATGCGTAAGGAGAAAATACCGCATCAGGCGTACT | |
| GTATACGCCACACTTTATGGCGTGTCTACGCATTCCTCTTTTATGGCGTAGTCCGCATGA | |
| 61 | |
| GAGTCATTAGGGACTTTCCAATGGGTTTTGCCCAGTACATAAGGTCAATAGGGGTGAATC | |
| CTCAGTAATCCCTGAAAGGTTACCCAAAACGGGTCATGTATTCCAGTTATCCCCACTTAG | |
| 121 | |
| AACAGGAAAGTCCCATTGGAGCCAAGTACACTGAGTCAATAGGGACTTTCCATTGGGTTT | |
| TTGTCCTTTCAGGGTAACCTCGGTTCATGTGACTCAGTTATCCCTGAAAGGTAACCCAAA | |
| 181 | |
| TGCCCAGTACAAAAGGTCAATAGGGGGTGAGTCAATGGGTTTTTCCCATTATTGGCACGT | |
| ACGGGTCATGTTTTCCAGTTATCCCCCACTCAGTTACCCAAAAAGGGTAATAACCGTGCA | |
| 241 | |
| ACATAAGGTCAATAGGGGTGAGTCATTGGGTTTTTCCAGCCAATTTAATTAAAACGCCAT | |
| TGTATTCCAGTTATCCCCACTCAGTAACCCAAAAAGGTCGGTTAAATTAATTTTGCGGTA | |
| 301 | |
| GTACTTTCCCACCATTGACGTCAATGGGCTATTGAAACTAATGCAACGTGACCTTTAAAC | |
| CATGAAAGGGTGGTAACTGCAGTTACCCGATAACTTTGATTACGTTGCACTGGAAATTTG | |
| 361 | |
| GGTACTTTCCCATAGCTGATTAATGGGAAAGTACCGTTCTCGAGCCAATACACGTCAATG | |
| CCATGAAAGGGTATCGACTAATTACCCTTTCATGGCAAGAGCTCGGTTATGTGCAGTTAC | |
| 421 | |
| GGAAGTGAAAGGGCAGCCAAAACGTAACACCGCCCCGGTTTTCCCCTGGAAATTCCATAT | |
| CCTTCACTTTCCCGTCGGTTTTGCATTGTGGCGGGGCCAAAAGGGGACCTTTAAGGTATA | |
| 481 | |
| TGGCACGCATTCTATTGGCTGAGCTGCGTTCTACGTGGGTATAAGAGGCGCGACCAGCGT | |
| ACCGTGCGTAAGATAACCGACTCGACGCAAGATGCACCCATATTCTCCGCGCTGGTCGCA | |
| 541 | |
| CGGTACCGTCGCAGTCTTCGGTCTGACCACCGTAGAACGCAGAGCTCCTCGCTGCAGCCC | |
| GCCATGGCAGCGTCAGAAGCCAGACTGGTGGCATCTTGCGTCTCGAGGAGCGACGTCGGG | |
| 601 | |
| AAGCTCTGTTGGGCTCGCGGTTGAGGACAAACTCTTCGCGGTCTTTCCAGTACTCTTGGA | |
| TTCGAGACAACCCGAGCGCCAACTCCTGTTTGAGAAGCGCCAGAAAGGTCATGAGAACCT | |
| 661 | |
| TCGGAAACCCGTCGGCCTCCGAACGGTACTCCGCCACCGAGGGACCTGAGCGAGTCCGCA | |
| AGCCTTTGGGCAGCCGGAGGCTTGCCATGAGGCGGTGGCTCCCTGGACTCGCTCAGGCGT | |
| 721 | |
| TCGACCGGATCGGAAAACCTCTCGACTGTTGGGGTGAGTACTCCCTCTCAAAAGCGGGCA | |
| AGCTGGCCTAGCCTTTTGGAGAGCTGACAACCCCACTCATGAGGGAGAGTTTTCGCCCGT | |
| 781 | |
| TGACTTCTGCGCTAAGATTGTCAGTTTCCAAAAACGAGGAGGATTTGATATTCACCTGGC | |
| ACTGAAGACGCGATTCTAACAGTCAAAGGTTTTTGCTCCTCCTAAACTATAAGTGGACCG | |
| 841 | |
| CCGCGGTGATGCCTTTGAGGGTGGCCGCGTCCATCTGGTCAGAAAAGACAATCTTTTTGT | |
| GGCGCCACTACGGAAACTCCCACCGGCGCAGGTAGACCAGTCTTTTCTGTTAGAAAAACA | |
| 901 | |
| TGTCAAGCTTGAGGTGTGGCAGGCTTGAGATCTGGCCATACACTTGAGTGACAATGACAT | |
| ACAGTTCGAACTCCACACCGTCCGAACTCTAGACCGGTATGTGAACTCACTGTTACTGTA | |
| 961 | |
| CCACTTTGCCTTTCTCTCCACAGGTGTCCACTCCCAGGTCCAACTGCAGGTCGACTCTAG | |
| GGTGAAACGGAAAGAGAGGTGTCCACAGGTGAGGGTCCAGGTTGACGTCCAGCTGAGATC | |
| 1021 (hIL-13Rα2.Fc coding region) | |
| CGCACCACCATGAAATTCTTAGTCAACGTTGCCCTTGTTTTTATGGTCGTGTACATTTCT | |
| GCGTGGTGGTACTTTAAGAATCAGTTGCAACGGGAACAAAAATACCAGCACATGTAAAGA | |
| P1 > M K F L V N V A L V F M V V Y I S | HBL |
| 1081 | |
| TACATCTATGCGACCGAGATAAAAGTTAACCCTCCTCAGGATTTTGAGATAGTGGATCCC | |
| ATGTAGATACGCTGGCTCTATTTTCAATTGGGAGGAGTCCTAAAACTCTATCACCTAGGG | |
| P18> Y I Y A T E I K V N P P Q D F E I V D P | hIL-13Rα2 |
| | extracellular |
| 1141 | domain |
| GGATACTTAGGTTATCTCTATTTGCAATGGCAACCCCCACTGTCTCTGGATCATTTTAAG | |
| CCTATGAATCCAATAGAGATAAACGTTACCGTTGGGGGTGACAGAGACCTAGTAAAATTC | |
| P38> G Y L G Y L Y L Q W Q P P L S L D H F K | |
| 1201 | |
| GAATGCACAGTGGAATATGAACTAAAATACCGAAACATTGGTAGTGAAACATGGAAGACC | |
| CTTACGTGTCACCTTATACTTGATTTTATGGCTTTGTAACCATCACTTTGTACCTTCTGG | |
| P58> E C T V E Y E L K Y R N I G S E T W K T | |
| 1261 | |
| ATCATTACTAAGAATCTACATTACAAAGATGGGTTTGATCTTAACAAGGGCATTGAAGCG | |
| TAGTAATGATTCTTAGATGTAATGTTTCTACCCAAACTAGAATTGTTCCCGTAACTTCGC | |
| P78> I I T K N L H Y K D G F D L N K G I E A | |
| 1321 | |
| AAGATACACACGCTTTTACCATGGCAATGCACAAATGGATCAGAAGTTCAAAGTTCCTGG | |
| TTCTATGTGTGCGAAAATGGTACCGTTACGTGTTTACCTAGTCTTCAAGTTTCAAGGACC | |
| P98> K I H T L L P W Q C T N G S E V Q S S W | |
| 1381 | |
| GCAGAAACTACTTATTGGATATCACCACAAGGAATTCCAGAAACTAAAGTTCAGGATATG | |
| CGTCTTTGATGAATAACCTATAGTGGTGTTCCTTAAGGTCTTTGATTTCAAGTCCTATAC | |
| P118> A E T T Y W I S P Q G I P E T K V Q D M | |
| 1441 | |
| GATTGCGTATATTACAATTGGCAATATTTACTCTGTTCTTGGAAACCTGGCATAGGTGTA | |
| CTAACGCATATAATGTTAACCGTTATAAATGAGACAAGAACCTTTGGACCGTATCCACAT | |
| P138> D C V Y Y N W Q Y L L C S W K P G I G V | |
| 1501 | |
| CTTCTTGATACCAATTACAACTTGTTTTACTGGTATGAGGGCTTGGATCATGCATTACAG | |
| GAAGAACTATGGTTAATGTTGAACAAAATGACCATACTCCCGAACCTAGTACGTAATGTC | |
| P158> L L D T N Y N L F Y W Y E G L D H A L Q | |
| 1561 | |
| TGTGTTGATTACATCAAGGCTGATGGACAAAATATAGGATGCAGATTTCCCTATTTGGAG | |
| ACACAACTAATGTAGTTCCGACTACCTGTTTTATATCCTACGTCTAAAGGGATAAACCTC | |
| P178> C V D Y I K A D G Q N I G C R F P Y L E | |
| 1621 | |
| GCATCAGACTATAAAGATTTCTATATTTGTGTTAATGGATCATCAGAGAACAAGCCTATC | |
| CGTAGTCTGATATTTCTAAAGATATAAACACAATTACCTAGTAGTCTCTTGTTCGGATAG | |
| P198> A S D Y K D F Y I C V N G S S E N K P I | |
| 1681 | |
| AGATCCAGTTATTTCACTTTTCAGCTTCAAAATATAGTTAAACCTTTGCCGCCAGTCTAT | |
| TCTAGGTCAATAAAGTGAAAAGTCGAAGTTTTATATCAATTTGGAAACGGCGGTCAGATA | |
| P218> R S S Y F T F Q L Q N I V K P L P P V Y | |
| 1741 | |
| CTTACTTTTACTCGGGAGAGTTCATGTGAAATTAAGCTGAAATGGAGCATACCTTTGGGA | |
| GAATGAAAATGAGCCCTCTCAAGTACACTTTAATTCGACTTTACCTCGTATGGAAACCCT | |
| P238> L T F T R E S S C E I K L K W S I P L G | |
| 1801 | |
| CCTATTCCAGCAAGGTGTTTTGATTATGAAATTGAGATCAGAGAAGATGATACTACCTTG | |
| GGATAAGGTCGTTCCACAAAACTAATACTTTAACTCTAGTCTCTTCTACTATGATGGAAC | |
| P258> P I P A R C F D Y E I E I R E D D T T L | |
| 1861 | |
| GTGACTGCTACAGTTGAAAATGAAACATACACCTTGAAAACAACAAATGAAACCCGACAA | |
| CACTGACGATGTCAACTTTTACTTTGTATGTGGAACTTTTGTTGTTTACTTTGGGCTGTT | |
| P278> V T A T V E N E T Y T L K T T N E T R Q | |
| 1921 | |
| TTATGCTTTGTAGTAAGAAGCAAAGTGAATATTTATTGCTCAGATGACGGAATTTGGAGT | |
| AATACGAAACATCATTCTTCGTTTCACTTATAAATAACGAGTCTACTGCCTTAAACCTCA | |
| P298> L C F V V R S K V N I Y C S D D G I W S | |
| 1981 | |
| GAGTGGAGTGATAAACAATGCTGGGAAGGTGAAGACCTATCGAAGAAAACTCCCAAATCT | |
| CTCACCTCACTATTTGTTACGACCCTTCCACTTCTGGATAGCTTCTTTTGAGGGTTTAGA | |
| P318> E W S D K Q C W E G E D L S K K T P K S | human IgG1 |
| | heavy chain |
| 2041 | |
| TGTGACAAAACTCACACATGCCCACCGTGCCCAGCACCTGAACTCCTGGGGGGACCGTCA | |
| ACACTGTTTTGAGTGTGTACGGGTGGCACGGGTCGTGGACTTGAGGACCCCCCTGGCAGT | |
| P338> C D K T H T C P P C P A P E L L G G P S | |
| 2101 | |
| GTCTTCCTCTTCCCCCCAAAACCCAAGGACACCCTCATGATCTCCCGGACCCCTGAGGTC | |
| CAGAAGGAGAAGGGGGGTTTTGGGTTCCTGTGGGAGTACTAGAGGGCCTGGGGACTCCAG | |
| P358> V F L F P P K P K D T L M I S R T P E V | |
| 2161 | |
| ACATGCGTGGTGGTGGACGTGAGCCACGAAGACCCTGAGGTCAAGTTCAACTGGTACGTG | |
| TGTACGCACCACCACCTGCACTCGGTGCTTCTGGGACTCCAGTTCAAGTTGACCATGCAC | |
| P378> T C V V V D V S H E D P E V K F N W Y V | |
| 2221 | |
| GACGGCGTGGAGGTGCATAATGCCAAGACAAAGCCGCGGGAGGAGCAGTACAACAGCACG | |
| CTGCCGCACCTCCACGTATTACGGTTCTGTTTCGGCGCCCTCCTCGTCATGTTGTCGTGC | |
| P398> D G V E V H N A K T K P R E E Q Y N S T | |
| 2281 | |
| TACCGTGTGGTCAGCGTCCTCACCGTCCTGCACCAGGACTGGCTGAATGGCAAGGAGTAC | |
| ATGGCACACCAGTCGCAGGAGTGGCAGGACGTGGTCCTGACCGACTTACCGTTCCTCATG | |
| P418> Y R V V S V L T V L H Q D W L N G K E Y | |
| 2341 | |
| AAGTGCAAGGTCTCCAACAAAGCCCTCCCAGTCCCCATCGAGAAAACCATCTCCAAAGCC | |
| TTCACGTTCCAGAGGTTGTTTCGGGAGGGTCAGGGGTAGCTCTTTTGGTAGAGGTTTCGG | |
| P438> K C K V S N K A L P V P I E K T I S K A | |
| 2401 | |
| AAAGGGCAGCCCCGAGAACCACAGGTGTACACCCTGCCCCCATCCCGGGAGGAGATGACC | |
| TTTCCCGTCGGGGCTCTTGGTGTCCACATGTGGGACGGGGGTAGGGCCCTCCTCTACTGG | |
| P458> K G Q P R E P Q V Y T L P P S R E E M T | |
| 2461 | |
| AAGAACCAGGTCAGCCTGACCTGCCTGGTCAAAGGCTTCTATCCCAGCGACATCGCCGTG | |
| TTCTTGGTCCAGTCGGACTGGACGGACCAGTTTCCGAAGATAGGGTCGCTGTAGCGGCAC | |
| P478> K N Q V S L T C L V K G F Y P S D I A V | |
| 2521 | |
| GAGTGGGAGAGCAATGGGCAGCCGGAGAACAACTACAAGACCACGCCTCCCGTGCTGGAC | |
| CTCACCCTCTCGTTACCCGTCGGCCTCTTGTTGATGTTCTGGTGCGGAGGGCACGACCTG | |
| P498> E W E S N G Q P E N N Y K T T P P V L D | |
| 2581 | |
| TCCGACGGCTCCTTCTTCCTCTATAGCAAGCTCACCGTGGACAAGAGCAGGTGGCAGCAG | |
| AGGCTGCCGAGGAAGAAGGAGATATCGTTCGAGTGGCACCTGTTCTCGTCCACCGTCGTC | |
| P518> S D G S F F L Y S K L T V D K S R W Q Q | |
| 2641 | |
| GGGAACGTCTTCTCATGCTCCGTGATGCATGAGGCTCTGCACAACCACTACACGCAGAAG | |
| CCCTTGCAGAAGAGTACGAGGCACTACGTACTCCGAGACGTGTTGGTGATGTGCGTCTTC | |
| P538> G N V F S C S V M H E A L H N H Y T Q K | |
| 2701 | |
| AGCCTCTCCCTGTCCCCGGGTAAATGAGTGAATTAATTCGGCGCGCCAAATTCTAACGTT | |
| TCGGAGAGGGACAGGGGCCCATTTACTCACTTAATTAAGCCGCGCGGTTTAAGATTGCAA | |
| P558> S L S L S P G K (SEQ ID NO:13) | |
| 2761 | |
| ACTGGCCGAAGCCGCTTGGAATAAGGCCGGTGTGCGTTTGTCTATATGTTATTTTCCACC | |
| TGACCGGCTTCGGCGAACCTTATTCCGGCCACACGCAAACAGATATACAATAAAAGGTGG | |
| 2821 | |
| ATATTGCCGTCTTTTGGCAATGTGAGGGCCCGGAAACCTGGCCCTGTCTTCTTGACGAGC | |
| TATAACGGCAGAAAACCGTTACACTCCCGGGCCTTTGGACCGGGACAGAAGAACTGCTCG | |
| 2881 | |
| ATTCCTAGGGGTCTTTCCCCTCTCGCCAAAGGAATGCAAGGTCTGTTGAATGTCGTGAAG | |
| TAAGGATCCCCAGAAAGGGGAGAGCGGTTTCCTTACGTTCCAGACAACTTACAGCACTTC | |
| 2941 | |
| GAAGCAGTTCCTCTGGAAGCTTCTTGAAGACAAACAACGTCTGTAGCGACCCTTTGCAGG | |
| CTTCGTCAAGGAGACCTTCGAAGAACTTCTGTTTGTTGCAGACATCGCTGGGAAACGTCC | |
| 3001 | |
| CAGCGGAACCCCCCACCTGGCGACAGGTGCCTCTGCGGCCAAAAGCCACGTGTATAAGAT | |
| GTCGCCTTGGGGGGTGGACCGCTGTCCACGGAGACGCCGGTTTTCGGTGCACATATTCTA | |
| 3061 | |
| ACACCTGCAAAGGCGGCACAACCCCAGTGCCACGTTGTGAGTTGGATAGTTGTGGAAAGA | |
| TGTGGACGTTTCCGCCGTGTTGGGGTCACGGTGCAACACTCAACCTATCAACACCTTTCT | |
| 3121 | |
| GTCAAATGGCTCTCCTCAAGCGTATTCAACAAGGGGCTGAAGGATGCCCAGAAGGTACCC | |
| CAGTTTACCGAGAGGAGTTCGCATAAGTTGTTCCCCGACTTCCTACGGGTCTTCCATGGG | |
| 3181 | |
| CATTGTATGGGATCTGATCTGGGGCCTCGGTGCACATGCTTTACATGTGTTTAGTCGAGG | |
| GTAACATACCCTAGACTAGACCCCGGAGCCACGTGTACGAAATGTACACAAATCAGCTCC | |
| 3241 | |
| TTAAAAAACGTCTAGGCCCCCCGAACCACGGGGACGTGGTTTTCCTTTGAAAAACACGAT | |
| AATTTTTTGCAGATCCGGGGGGCTTGGTGCCCCTGCACCAAAAGGAAACTTTTTGTGCTA | |
| 3301 (DHFR coding region) | |
| TGCTCGAGCCATCATGGTTCGACCATTGAACTGCATCGTCGCCGTGTCCCAAAATATGGG | |
| ACGAGCTCGGTAGTACCAAGCTGGTAACTTGACGTAGCAGCGGCACAGGGTTTTATACCC | |
| > M V R P L N C I V A V S Q N M G | |
| 3361 | |
| GATTGGCAAGAACGGAGACCTACCCTGGCCTCCGCTCAGGAACGAGTTCAAGTACTTCCA | |
| CTAACCGTTCTTGCCTCTGGATGGGACCGGAGGCGAGTCCTTGCTCAAGTTCATGAAGGT | |
| > I G K N G D L P W P P L R N E F K Y F Q | |
| 3421 | |
| AAGAATGACCACAACCTCTTCAGTGGAAGGTAAACAGAATCTGGTGATTATGGGTAGGAA | |
| TTCTTACTGGTGTTGGAGAAGTCACCTTCCATTTGTCTTAGACCACTAATACCCATCCTT | |
| > R M T T T S S V E G K Q N L V I M G R K | |
| 3481 | |
| AACCTGGTTCTCCATTCCTGAGAAGAATCGACCTTTAAAGGACAGAATTAATATAGTTCT | |
| TTGGACCAAGAGGTAAGGACTCTTCTTAGCTGGAAATTTCCTGTCTTAATTATATCAAGA | |
| > T W F S I P E K N R P L K D R I N I V L | |
| 3541 | |
| CAGTAGAGAACTCAAAGAACCACCACGAGGAGCTCATTTTCTTGCCAAAAGTTTGGATGA | |
| GTCATCTCTTGAGTTTCTTGGTGGTGCTCCTCGAGTAAAAGAACGGTTTTCAAACCTACT | |
| > S R E L K E P P R G A H F L A K S L D D | |
| 3601 | |
| TGCCTTAAGACTTATTGAACAACCGGAATTGGCAAGTAAAGTAGACATGGTTTGGATAGT | |
| ACGGAATTCTGAATAACTTGTTGGCCTTAACCGTTCATTTCATCTGTACCAAACCTATCA | |
| > A L R L I E Q P E L A S K V D M V W I V | |
| 3661 | |
| CGGAGGCAGTTCTGTTTACCAGGAAGCCATGAATCAACCAGGCCACCTCAGACTCTTTGT | |
| GCCTCCGTCAAGACAAATGGTCCTTCGGTACTTAGTTGGTCCGGTGGAGTCTGAGAAACA | |
| > G G S S V Y Q E A M N Q P G H L R L F V | |
| 3721 | |
| GACAAGGATCATGCAGGAATTTGAAAGTGACACGTTTTTCCCAGAAATTGATTTGGGGAA | |
| CTGTTCCTAGTACGTCCTTAAACTTTCACTGTGCAAAAAGGGTCTTTAACTAAACCCCTT | |
| > T R I M Q E F E S D T F F P E I D L G K | |
| 3781 | |
| ATATAAACTTCTCCCAGAATACCCAGGCGTCCTCTCTGAGGTCCAGGAGGAAAAAGGCAT | |
| TATATTTGAAGAGGGTCTTATGGGTCCGCAGGAGAGACTCCAGGTCCTCCTTTTTCCGTA | |
| > Y K L L P E Y P G V L S E V Q E E K G I | |
| 3841 | |
| CAAGTATAAGTTTGAAGTCTACGAGAAGAAAGACTAACAGGAAGATGCTTTCAAGTTCTC | |
| GTTCATATTCAAACTTCAGATGCTCTTCTTTCTGATTGTCCTTCTACGAAAGTTCAAGAG | |
| > K Y K F E V Y E K K D (SEQ ID NO:14) | |
| 3901 | |
| TGCTCCCCTCCTAAAGCTATGCATTTTTTATAAGACCATGGGACTTTTGCTGGCTTTAGA | |
| ACGAGGGGAGGATTTCGATACGTAAAAAATATTCTGGTACCCTGAAAACGACCGAAATCT | |
| 3961 | |
| TCATAATCAGCCATACCACATTTGTAGAGGTTTTACTTGCTTTAAAAAACCTCCCACACC | |
| AGTATTAGTCGGTATGGTGTAAACATCTCCAAAATGAACGAAATTTTTTGGAGGGTGTGG | |
| 4021 | |
| TCCCCCTGAACCTGAAACATAAAATGAATGCAATTGTTGTTGTTAACTTGTTTATTGCAG | |
| AGGGGGACTTGGACTTTGTATTTTACTTACGTTAACAACAACAATTGAACAAATAACGTC | |
| 4081 | |
| CTTATAATGGTTACAAATAAAGCAATAGCATCACAAATTTCACAAATAAAGCATTTTTTT | |
| GAATATTACCAATGTTTATTTCGTTATCGTAGTGTTTAAAGTGTTTATTTCGTAAAAAAA | |
| 4141 | |
| CACTGCATTCTAGTTGTGGTTTGTCCAAACTCATCAATGTATCTTATCATGTCTGGATCC | |
| GTGACGTAAGATCAACACCAAACAGGTTTGAGTAGTTACATAGAATAGTACAGACCTAGG | |
| 4201 | |
| CCGGCCAACGGTCTGGTGACCCGGCTGCGAGAGCTCGGTGTACCTGAGACGCGAGTAAGC | |
| GGCCGGTTGCCAGACCACTGGGCCGACGCTCTCGAGCCACATGGACTCTGCGCTCATTCG | |
| 4261 | |
| CCTTGAGTCAAAGACGTAGTCGTTGCAAGTCCGCACCAGGTACTGATCATCGATGCTAGA | |
| GGAACTCAGTTTCTGCATCAGCAACGTTCAGGCGTGGTCCATGACTAGTAGCTACGATCT | |
| 4321 | |
| CCGTGCAAAAGGAGAGCCTGTAAGCGGGCACTCTTCCGTGGTCTGGTGGATAAATTCGCA | |
| GGCACGTTTTCCTCTCGGACATTCGCCCGTGAGAAGGCACCAGACCACCTATTTAAGCGT | |
| 4381 | |
| AGGGTATCATGGCGGACGACCGGGGTTCGAACCCCGGATCCGGCCGTCCGCCGTGATCCA | |
| TCCCATAGTACCGCCTGCTGGCCCCAAGCTTGGGGCCTAGGCCGGCAGGCGGCACTAGGT | |
| 4441 | |
| TCCGGTTACCGCCCGCGTGTCGAACCCAGGTGTGCGACGTCAGACAACGGGGGAGCGCTC | |
| AGGCCAATGGCGGGCGCACAGCTTGGGTCCACACGCTGCAGTCTGTTGCCCCCTCGCGAG | |
| 4501 | |
| CTTTTGGCTTCCTTCCAGGCGCGGCGGCTGCTGCGCTAGCTTTTTTGGCGAGCTCGAATT | |
| GAAAACCGAAGGAAGGTCCGCGCCGCCGACGACGCGATCGAAAAAACCGCTCGAGCTTAA | |
| 4561 | |
| AATTCTGCATTAATGAATCGGCCAACGCGCGGGGAGAGGCGGTTTGCGTATTGGGCGCTC | |
| TTAAGACGTAATTACTTAGCCGGTTGCGCGCCCCTCTCCGCCAAACGCATAACCCGCGAG | |
| 4621 | |
| TTCCGCTTCCTCGCTCACTGACTCGCTGCGCTCGGTCGTTCGGCTGCGGCGAGCGGTATC | |
| AAGGCGAAGGAGCGAGTGACTGAGCGACGCGAGCCAGCAAGCCGACGCCGCTCGCCATAG | |
| 4681 | |
| AGCTCACTCAAAGGCGGTAATACGGTTATCCACAGAATCAGGGGATAACGCAGGAAAGAA | |
| TCGAGTGAGTTTCCGCCATTATGCCAATAGGTGTCTTAGTCCCCTATTGCGTCCTTTCTT | |
| 4741 | |
| CATGTGAGCAAAAGGCCAGCAAAAGGCCAGGAACCGTAAAAAGGCCGCGTTGCTGGCGTT | |
| GTACACTCGTTTTCCGGTCGTTTTCCGGTCCTTGGCATTTTTCCGGCGCAACGACCGCAA | |
| 4801 | |
| TTTCCATAGGCTCCGCCCCCCTGACGAGCATCACAAAAATCGACGCTCAAGTCAGAGGTG | |
| AAAGGTATCCGAGGCGGGGGGACTGCTCGTAGTGTTTTTAGCTGCGAGTTCAGTCTCCAC | |
| 4861 | |
| GCGAAACCCGACAGGACTATAAAGATACCAGGCGTTTCCCCCTGGAAGCTCCCTCGTGCG | |
| CGCTTTGGGCTGTCCTGATATTTCTATGGTCCGCAAAGGGGGACCTTCGAGGGAGCACGC | |
| 4921 | |
| CTCTCCTGTTCCGACCCTGCCGCTTACCGGATACCTGTCCGCCTTTCTCCCTTCGGGAAG | |
| GAGAGGACAAGGCTGGGACGGCGAATGGCCTATGGACAGGCGGAAAGAGGGAAGCCCTTC | |
| 4981 | |
| CGTGGCGCTTTCTCAATGCTCACGCTGTAGGTATCTCAGTTCGGTGTAGGTCGTTCGCTC | |
| GCACCGCGAAAGAGTTACGAGTGCGACATCCATAGAGTCAAGCCACATCCAGCAAGCGAG | |
| 5041 | |
| CAAGCTGGGCTGTGTGCACGAACCCCCCGTTCAGCCCGACCGCTGCGCCTTATCCGGTAA | |
| GTTCGACCCGACACACGTGCTTGGGGGGCAAGTCGGGCTGGCGACGCGGAATAGGCCATT | |
| 5101 | |
| CTATCGTCTTGAGTCCAACCCGGTAAGACACGACTTATCGCCACTGGCAGCAGCCACTGG | |
| GATAGCAGAACTCAGGTTGGGCCATTCTGTGCTGAATAGCGGTGACCGTCGTCGGTGACC | |
| 5161 | |
| TAACAGGATTAGCAGAGCGAGGTATGTAGGCGGTGCTACAGAGTTCTTGAAGTGGTGGCC | |
| ATTGTCCTAATCGTCTCGCTCCATACATCCGCCACGATGTCTCAAGAACTTCACCACCGG | |
| 5221 | |
| TAACTACGGCTACACTAGAAGGACAGTATTTGGTATCTGCGCTCTGCTGAAGCCAGTTAC | |
| ATTGATGCCGATGTGATCTTCCTGTCATAAACCATAGACGCGAGACGACTTCGGTCAATG | |
| 5281 | |
| CTTCGGAAAAAGAGTTGGTAGCTCTTGATCCGGCAAACAAACCACCGCTGGTAGCGGTGG | |
| GAAGCCTTTTTCTCAACCATCGAGAACTAGGCCGTTTGTTTGGTGGCGACCATCGCCACC | |
| 5341 | |
| TTTTTTTGTTTGCAAGCAGCAGATTACGCGCAGAAAAAAAGGATCTCAAGAAGATCCTTT | |
| AAAAAAACAAACGTTCGTCGTCTAATGCGCGTCTTTTTTTCCTAGAGTTCTTCTAGGAAA | |
| 5401 | |
| GATCTTTTCTACGGGGTCTGACGCTCAGTGGAACGAAAACTCACGTTAAGGGATTTTGGT | |
| CTAGAAAAGATGCCCCAGACTGCGAGTCACCTTGCTTTTGAGTGCAATTCCCTAAAACCA | |
| 5461 | |
| CATGAGATTATCAAAAAGGATCTTCACCTAGATCCTTTTAAATTAAAAATGAAGTTTTAA | |
| GTACTCTAATAGTTTTTCCTAGAAGTGGATCTAGGAAAATTTAATTTTTACTTCAAAATT | |
| 5521 | |
| ATCAATCTAAAGTATATATGAGTAAACTTGGTCTGACAGTTACCAATGCTTAATCAGTGA | |
| TAGTTAGATTTCATATATACTCATTTGAACCAGACTGTCAATGGTTACGAATTAGTCACT | |
| 5581 | |
| GGCACCTATCTCAGCGATCTGTCTATTTCGTTCATCCATAGTTGCCTGACTCCCCGTCGT | |
| CCGTGGATAGAGTCGCTAGACAGATAAAGCAAGTAGGTATCAACGGACTGAGGGGCAGCA | |
| 5641 | |
| GTAGATAACTACGATACGGGAGGGCTTACCATCTGGCCCCAGTGCTGCAATGATACCGCG | |
| CATCTATTGATGCTATGCCCTCCCGAATGGTAGACCGGGGTCACGACGTTACTATGGCGC | |
| 5701 | |
| AGACCCACGCTCACCGGCTCCAGATTTATCAGCAATAAACCAGCCAGCCGGAAGGGCCGA | |
| TCTGGGTGCGAGTGGCCGAGGTCTAAATAGTCGTTATTTGGTCGGTCGGCCTTCCCGGCT | |
| 5761 | |
| GCGCAGAAGTGGTCCTGCAACTTTATCCGCCTCCATCCAGTCTATTAATTGTTGCCGGGA | |
| CGCGTCTTCACCAGGACGTTGAAATAGGCGGAGGTAGGTCAGATAATTAACAACGGCCCT | |
| 5821 | |
| AGCTAGAGTAAGTAGTTCGCCAGTTAATAGTTTGCGCAACGTTGTTGCCATTGCTACAGG | |
| TCGATCTCATTCATCAAGCGGTCAATTATCAAACGCGTTGCAACAACGGTAACGATGTCC | |
| 5881 | |
| CATCGTGGTGTCACGCTCGTCGTTTGGTATGGCTTCATTCAGCTCCGGTTCCCAACGATC | |
| GTAGCACCACAGTGCGAGCAGCAAACCATACCGAAGTAAGTCGAGGCCAAGGGTTGCTAG | |
| 5941 | |
| AAGGCGAGTTACATGATCCCCCATGTTGTGCAAAAAAGCGGTTAGCTCCTTCGGTCCTCC | |
| TTCCGCTCAATGTACTAGGGGGTACAACACGTTTTTTCGCCAATCGAGGAAGCCAGGAGG | |
| 6001 | |
| GATCGTTGTCAGAAGTAAGTTGGCCGCAGTGTTATCACTCATGGTTATGGCAGCACTGCA | |
| CTAGCAACAGTCTTCATTCAACCGGCGTCACAATAGTGAGTACCAATACCGTCGTGACGT | |
| 6061 | |
| TAATTCTCTTACTGTCATGCCATCCGTAAGATGCTTTTCTGTGACTGGTGAGTACTCAAC | |
| ATTAAGAGAATGACAGTACGGTAGGCATTCTACGAAAAGACACTGACCACTCATGAGTTG | |
| 6121 | |
| CAAGTCATTCTGAGAATAGTGTATGCGGCGACCGAGTTGCTCTTGCCCGGCGTCAATACG | |
| GTTCAGTAAGACTCTTATCACATACGCCGCTGGCTCAACGAGAACGGGCCGCAGTTATGC | |
| 6181 | |
| GGATAATACCGCGCCACATAGCAGAACTTTAAAAGTGCTCATCATTGGAAAACGTTCTTC | |
| CCTATTATGGCGCGGTGTATCGTCTTGAAATTTTCACGAGTAGTAACCTTTTGCAAGAAG | |
| 6241 | |
| GGGGCGAAAACTCTCAAGGATCTTACCGCTGTTGAGATCCAGTTCGATGTAACCCACTCG | |
| CCCCGCTTTTGAGAGTTCCTAGAATGGCGACAACTCTAGGTCAAGCTACATTGGGTGAGC | |
| 6301 | |
| TGCACCCAACTGATCTTCAGCATCTTTTACTTTCACCAGCGTTTCTGGGTGAGCAAAAAC | |
| ACGTGGGTTGACTAGAAGTCGTAGAAAATGAAAGTGGTCGCAAAGACCCACTCGTTTTTG | |
| 6361 | |
| AGGAAGGCAAAATGCCGCAAAAAAGGGAATAAGGGCGACACGGAAATGTTGAATACTCAT | |
| TCCTTCCGTTTTACGGCGTTTTTTCCCTTATTCCCGCTGTGCCTTTACAACTTATGAGTA | |
| 6421 | |
| ACTCTTCCTTTTTCAATATTATTGAAGCATTTATCAGGGTTATTGTCTCATGAGCGGATA | |
| TGAGAAGGAAAAAGTTATAATAACTTCGTAAATAGTCCCAATAACAGAGTACTCGCCTAT | |
| 6481 | |
| CATATTTGAATGTATTTAGAAAAATAAACAAATAGGGGTTCCGCGCACATTTCCCCGAAA | |
| GTATAAACTTACATAAATCTTTTTATTTGTTTATCCCCAAGGCGCGTGTAAAGGGGCTTT | |
| 6541 | |
| AGTGCCACCTGACGTCTAAGAAACCATTATTATCATGACATTAACCTATAAAAATAGGCG | |
| TCACGGTGGACTGCAGATTCTTTGGTAATAATAGTACTGTAATTGGATATTTTTATCCGC | |
| 6601 | |
| TATCACGAGGCCCTTTCGTCTCGCGCGTTTCGGTGATGACGGTGAAAACCTCTGACACAT | |
| ATAGTGCTCCGGGAAAGCAGAGCGCGCAAAGCCACTACTGCCACTTTTGGAGACTGTGTA | |
| 6661 | |
| GCAGCTCCCGGAGACGGTCACAGCTTGTCTGTAAGCGGATGCCGGGAGCAGACAAGCCCG | |
| CGTCGAGGGCCTCTGCCAGTGTCGAACAGACATTCGCCTACGGCCCTCGTCTGTTCGGGC | |
| 6721 | |
| TCAGGGCGCGTCAGCGGGTGTTGGCGGGTGTCGGGGCTGGCTTAACTATGCGGCATCAGA | |
| AGTCCCGCGCAGTCGCCCACAACCGCCCACAGCCCCGACCGAATTGATACGCCGTAGTCT | |
| 6781 | |
| GCAGATTGTACTGAGAGTGCAC (SEQ ID NO:15) | |
| CGTCTAACATGACTCTCACGTG (SEQ ID NO:16) | |

### Example 2

### Transient Co-Transfection of COS Cells with Plasmids Encoding a Soluble IL-13 Antagonist, Human IL-13Rα2.Fc, and Human IL-13 Increases the Level of IL13Rα2Fc Expression

The effect of hIL-13 on hIL-13Rα2.Fc encoded by L2I expression vector was assessed in a COS cellular expression system. Presented below are the results of enzyme-linked immunoassay (ELISA) results of the conditioned media of transiently transfected COS cells.

| | PMR159 |
|---|---|
| Treatment | (μg/ml) |
| MOCK | 0 |
| L2I | 0.39 |
| L2I + pED | 0.52 |
| L2I + IL-13 (pXMT2 (DD)) | 3.35 |
| L2I + IL-13 (pXMT2 (PMR)) | 3.93 |
| L2I + IL-13 (pEMC3 (SK)) | 1.25 |
| L2I + IL-13 (1 μg/ml rE:coli hIL-13 (R&D)) | 0.38 |
| L2I + IL-13 (1 μg/ml rCHOmIL-13 (DD)) | 0.45 |

No hIL-13Rα2.Fc polypeptide was detected in mock transfected cells. Co-transfection of L2I with each of three different hIL-13 expression plasmids (i.e., pXMT2 (DD); pXMT2 (PMR); pEMC3 (SK)) resulted in hIL-13Rα2.Fc polypeptide expression (1.25 μg/ml to 3.93 μg/ml) significantly higher than the level of IL-13Rα2.Fc polypeptide production observed in either the L2I+pED vector treatment group (0.52 μg/ml) or L2I control (0.39 μg/ml).

Adding exogenous hIL-13 (1 μg/ml) derived from either a hIL-13-expressing recombinant E. coli strain (rE:coli hIL-13 (R&D)) or an IL-13-expressing CHO cell line (rCHOmIL-13 (DD)) to cells transfected with L2I did not significantly increase hIL-13Rα2.Fc polypeptide production compared with the level of hIL-13Rα2.Fc polypeptide production in the L2I+pED vector control (0.52 μg/ml). This result demonstrates that hIL-13 affects the level of hIL-13Rα2.Fc fusion polypeptide accumulated in the conditioned medium by an interaction in the process of synthesis and secretion of the Fc fusion polypeptide, and not by an interaction outside the cell.

Levels of nascent hIL-13Rα2.Fc in COS cells co-transfected with both L2I and hIL-13 were similar to the level of nascent IL-13 Rα1.Fc, even though the latter fusion polypeptide normally shows 20-fold higher accumulation in conditioned medium relative to hIL-13Rα2.Fc. The defect in hIL-13Rα2.Fc secretion appears to be corrected by co-expression with hIL-13. Although not wishing to be bound by theory, the results could be explained by showing that the hIL-13Rα2.Fc-IL-13 complex is more efficiently secreted by cells than hIL-13Rα2.Fc alone.

As summarized below, subsequent studies corroborated the enhancement of hIL-13Rα2.Fc polypeptide production when hIL-13 was co-expressed with hIL-13Rα2.Fc polypeptide in the COS cell expression system.

| Treatment | PMR162 (μg/ml) | PMR164 (μg/ml) |
|---|---|---|
| MOCK | ND | ND |
| IL-13 + pED | 0 | 0 |
| L2I + pED | 0.543 | 0.472 |
| L2I + IL-13 (pXMT2 (PMR)) | 3.32 | 4.63 |
| L2I + IL-6 | 1.44 | 1.22 |
| L2I + M-CSF | 0.863 | 0.858 |

The effect of hIL-13Rα2.Fc polypeptide production in media from cells transfected with pL2I and non-IL-13 receptor ligands was also examined. Co-transfection of L2I plasmid and a plasmid directing expression of hIL-6 (1.2-1.3 μg/ml) or a plasmid directing the production of M-CSF (˜0.86 μg/ml) yielded elevated production of the hIL-13Rα2.Fc polypeptide compared to the production level of the fusion polypeptide detected in cells transfected with L2I+pED vector (˜0.5 μg/ml). The effect of the hIL-6 and M-CFS polypeptide expression on hIL-13Rα2.Fc polypeptide production was, however, less than the ˜6 to 9-fold elevation of hIL-13Rα2.Fc polypeptide production observed in cells co-expressing the hIL-13 ligand (3.32-4.6 μg/ml).

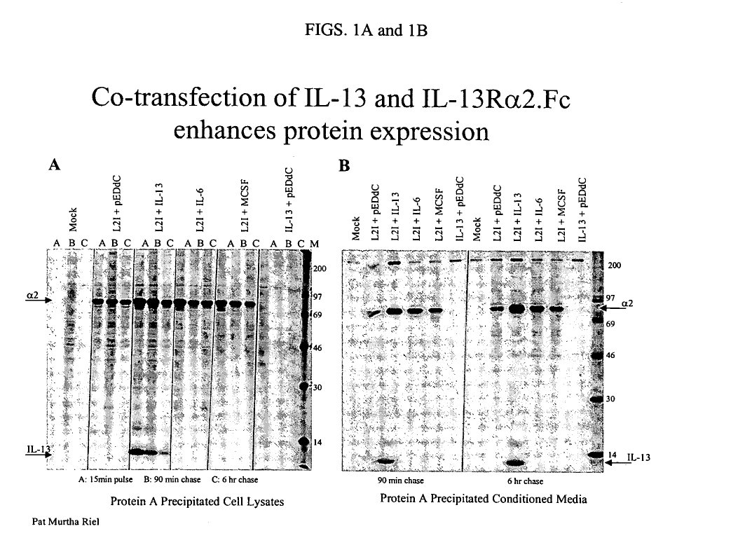

The accumulated hIL-13Rα2.Fc fusion polypeptide in the medium of transfected COS cells was also examined by pulse-chase radiolabeling of the transfected COS cells. Transfected COS cells were radiolabeled by synthetic incorporation of ³⁵S methionine and cysteine in a 15 minute pulse. Samples were analyzed by SDS PAGE and the ³⁵S-protein was then visualized using autoradiography. Analysis of the total conditioned medium of cells is shown in FIG. 1A. Analysis of radiolabeled hIL-13Rα2.Fc fusion polypeptide concentrated from the total media by protein A precipitation prior to SDS PAGE and autoradiograph is shown in FIG. 1B. Consistent with the ELISA data, an increased level of fusion polypeptide was detected in the conditioned medium of cells co-transfected with L2I+hIL-13 encoding plasmids relative to cells co-transfected with L2I plasmid, hIL-13 plasmid, or cells co-transfected with L2I+hIL-6, or L2I+M-CSF.

### Example 3

### Stable Co-Transfection of CHO Cells with Plasmids Encoding A Soluble IL-13 Antagonist, IL-13Rα2.Fc, and IL-13 Increase the Level of IL-13Rα2.Fc Expression

Studies of IL-13Rα2.Fc fusion polypeptide expression using COS cell transient transfection assays (Example 1) were extended using stable CHO cell lines expressing hIL-13Rα2.Fc fusion polypeptide.

A. Preparation of Stable CHO Cells Co-Expressing hIL-13Rα2.Fc Fusion Polypeptide and hIL-13 Polypeptide

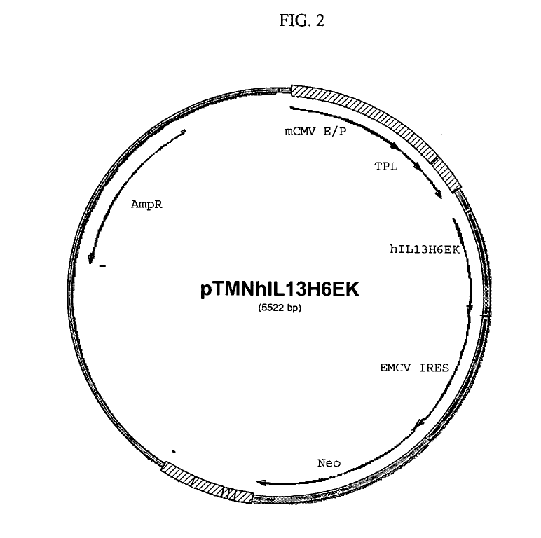

A stable hIL-13Rα2.Fc fusion polypeptide expressing CHO cell line was stably transfected with an expression plasmid containing the hIL-13 gene and the neomycin resistance marker (FIG. 2). As detailed in FIG. 2, transcription of the hIL-13 expression plasmid pTMNhIL13H6EK was driven by the enhancer and promoter sequences derived from mouse cytomegalovirus (mCMV). The tripartite leader (TPL) sequence from the adenovirus major late promoter enhanced the translational efficiency. The hIL-13 coding region was cloned in-frame with a 6x-His tag to allow for one-step purification of the protein on a metal affinity column. The enterokinase cleavage site was engineered between the 6x-His tag and the hIL-13 coding region to allow post-purification removal of the 6x-His tag. The hIL-13 gene was expressed as part of a bicistronic message with the neomycin resistance (neo^{R}) marker. Translation of the neo^{R} gene was mediated from the encephalomyocarditis viral internal ribosome entry site (EMCV IRES). Following transfection, cells expressing hIL-13 were selected by culturing in the presence of the antibiotic G418.

B. Co-Expression of hIL-13Rα2.Fc Fusion Polypeptide and hIL-13 Enhances the Expression of hIL-13Rα2.Fc Fusion Polypeptide in CHO Cells

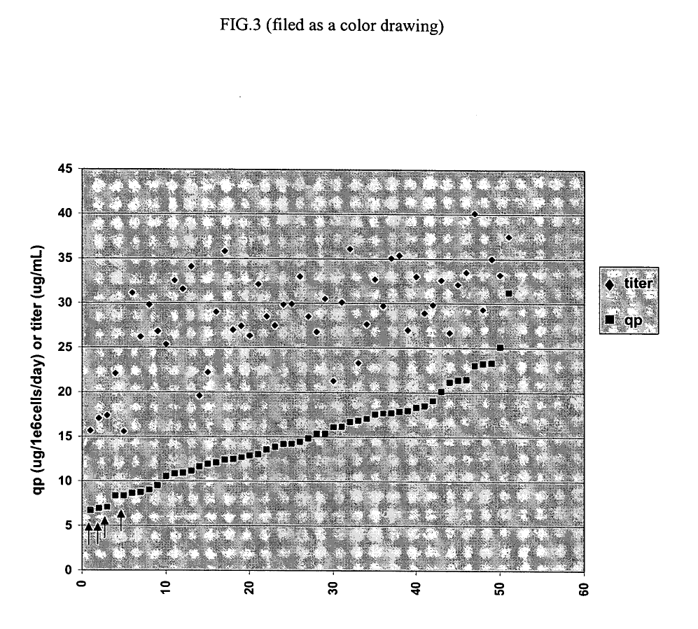

Like the COS cell system, expression of hIL-13Rα2.Fc fusion polypeptide in the hIL-13 co-expressing CHO clones was compared against the CHO cell line expressing hIL-13Rα2.Fc fusion polypeptide alone (FIG. 3). A stable cell line expressing hIL-13Rα2.Fc fusion polypeptide (6FD3) was transfected with the pTMNHIL13H6EK plasmid, and cells expressing hIL-13 were selected for by growth in medium containing the antibiotic G418. Clones were picked and assayed in a 7-day secretion assay at 31° C., and titers were measured by Protein A-HPLC. The results are shown in FIG. 3, where the productivities of four 6FD3 controls are designated by arrows and all other data points represent individual clones of hIL-13 co-expressing cells. As detailed in the Figure, all of the clones that were analyzed had higher expression levels of hIL-13Rα2.Fc fusion polypeptide than the parent cell line. Western blot analysis confirmed that the clones express hIL-13. Expression of hIL-13Rα2.Fc fusion polypeptide in an hIL-13 co-expressing cell line (31B5) at 37° C. was also assessed in a 14-day fed-batch assay.

C. Growing CHO Cells that Co-Express hIL-13Rα2.Fc Fusion Polypeptide and hIL-13 at Reduced Temperature Improve the Production of hIL-13Rα2.Fc Fusion Polypeptide

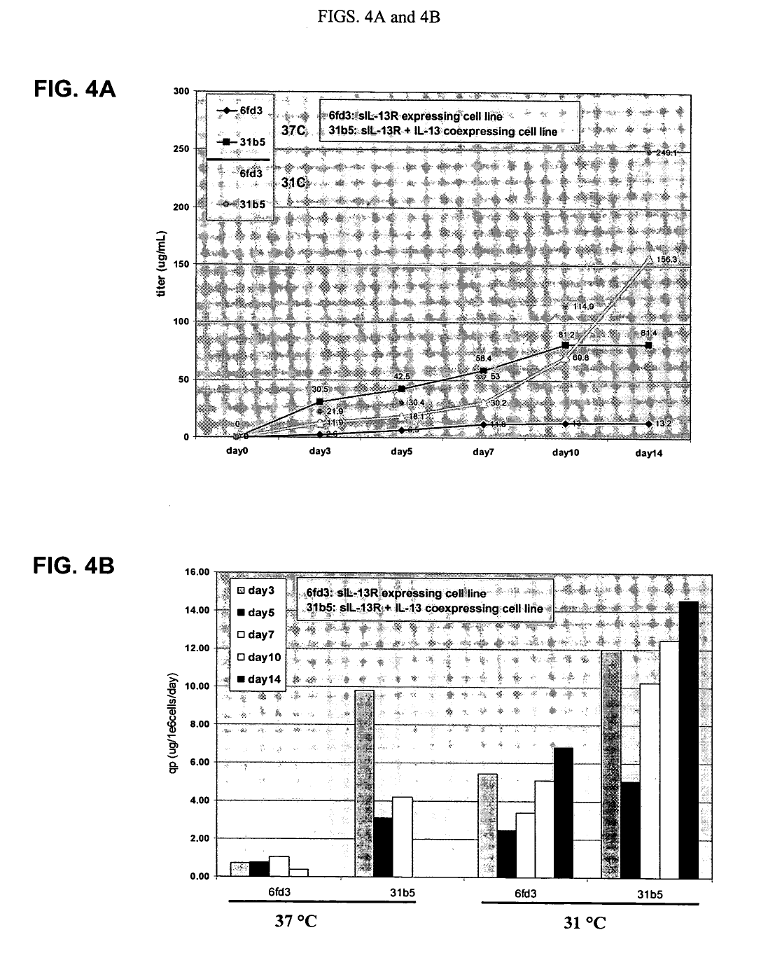

The effect of temperature on the expression of hIL-13Rα2.Fc fusion polypeptide was assessed in 6FD3 parental cells and hIL-13 co-expressing cell line 31B5 in a 14-day fed-batch assay. As shown in FIG. 4A, a time-dependent increase in hIL-13Rα2.Fc fusion polypeptide was observed over the 14-day study period in both 6FD3 parental cells and hIL-13 co-expressing cell line 31B5. Further, at both 37° C. and 31° C., the 31B5 cell line co-expressing the hIL-13Rα2.Fc fusion polypeptide and hIL-13 expressed higher level of hIL-13Rα2.Fc fusion polypeptide than the 6FD3 parental cell line. As shown in FIG. 4B, the specific cellular productivity of the hIL-13Rα2.Fc fusion polypeptide in the 31B5 co-expressing cell line was higher than the 6FD3 parent cell line. Moreover, the productivity of cells grown at 31° C. was higher than the productivity of cells grown at 37° C. That is, the CHO 31B5 co-expressing cells cultured at 31° C. exhibit significantly higher levels of hIL-13Rα2.Fc fusion polypeptide expression and/or secretion into the conditioned medium compared to the hIL-13Rα2.Fc fusion polypeptide expression observed when these cells are grown at 37° C.

D. Co-Expressing hIL-13Rα2.Fc Fusion Polypeptide and hIL-13 Reduces Molecular Aggregation of hIL-13Rα2.Fc Fusion Polypeptide

The expression level of soluble IL-13 antagonist, hIL-13Rα2.Fc is low due to molecular aggregation. The effect of co-expressing hIL-13 on the molecular aggregation of hIL-13Rα2.Fc fusion polypeptide was assessed by comparing the molecular aggregation state of the hIL-13Rα2.Fc fusion polypeptide in the medium of 31B5 cell line co-expressing the hIL-13Rα2.Fc fusion polypeptide and hIL-13 with the molecular aggregation state of hIL-13Rα2.Fc fusion polypeptide produced by the 6FD3 parental cell line using size exclusion chromatography HPLC (SEC-HPLC). Briefly, cell culture media from test cell lines was harvested and prepared for SEC-HPLC by purifying the samples on Protein A Sepharose beads.

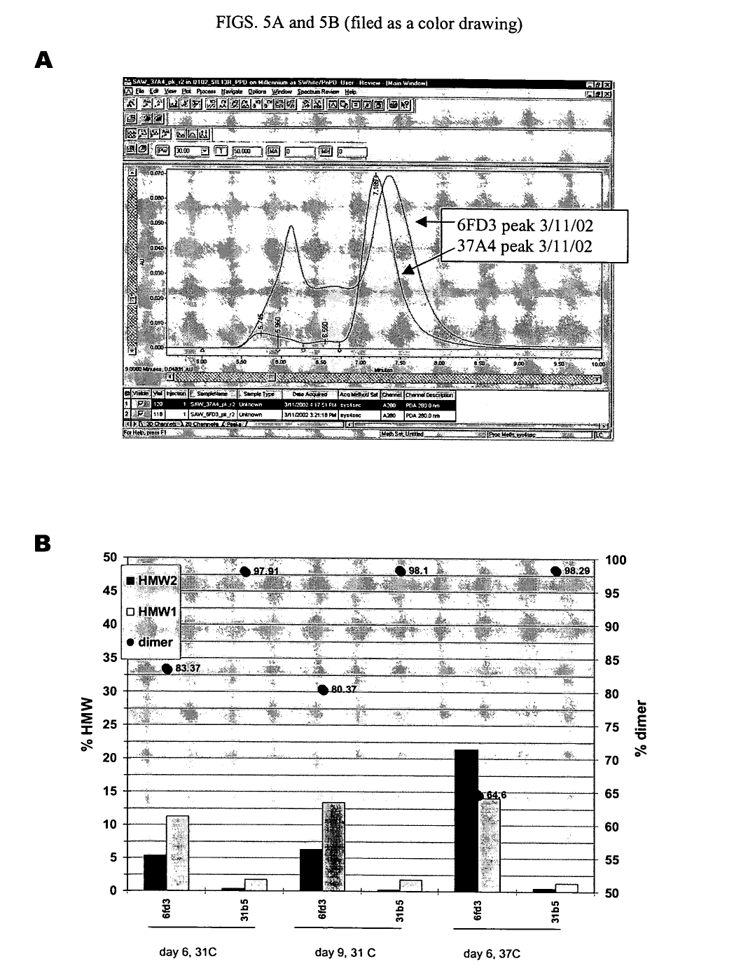

An overlay of the SEC-HPLC chromatogram of sample from the 37A4 cell line co-expressing the hIL-13Rα2.Fc fusion polypeptide and hIL-13 and the chromatogram of sample from the 6FD3 parental cell line revealed the relative distribution of dimer and high molecular weight species represented from the two cell lines (FIG. 5A). As shown in FIG. 5A, a typical chromatograph of hIL-13Rα2.Fc fusion polypeptide containing conditioned medium obtained from 6FD3 parental cell line showed multiple peaks of hIL-13Rα2.Fc fusion polypeptide, e.g., peak retention time=˜6.1-6.7 minutes, which represent high molecular weight species relative to the hIL-13Rα2.Fc fusion polypeptide dimer (peak retention time =7.2 minutes). In contrast, the SEC profile generated from the 31 B5 hIL-13 co-expressing cell line showed much reduced peaks of high molecular weight species relative to the dimer peak (peak retention time=˜7.4 minutes).

The low levels of aggregate found in the conditioned medium of the hIL-13 co-expressing cell line were maintained over long culture periods, and were observed when hIL-13Rα2.Fc fusion polypeptide-producing cells were grown at either 31° C. or 37° C. (FIG. 5B). The relative distribution of dimer and high molecular weight species represented in SEC-HPLC chromatograms of sample from the 31B5 cell line co-expressing the hIL-13Rα2.Fc fusion polypeptide and hIL-13 and the chromatogram of sample from the 6FD3 parental cell line were compared. The chromatograms of hIL-13Rα2.Fc fusion polypeptide containing conditioned medium obtained from 6FD3 parental cell line showed three major peaks. Two peaks, designated as HMW1 and HMW2, precede a peak containing dimerized hIL-13Rα2.Fc fusion polypeptide. That is, the peak that eluted first (retention time=˜8.2 min) was designated “HMW2”, the second peak (retention time=˜8.4-8.6 minutes) was designated “HMW1”, and the third peak (retention time=˜9.4-9.7 minutes) represented the hIL-13Rα2.Fc fusion polypeptide dimer. In contrast, the SEC profile generated from the 31B5hIL-13 co-expressing cell line showed much reduced HMW1 and HMW2 peaks relative to the dimer peak.

As shown in FIG. 5B, the relative percentages of each of the major hIL-13Rα2.Fc fusion polypeptide species present in conditioned medium on days 6 and 9 at 31° C. or on day 6 at 37° C. did not change significantly between day 6 and day 9 of cell culture. Likewise, growth temperature did not appear to significantly affect the molecular aggregation state of the hIL-13Rα2.Fc fusion polypeptide over the study period.

E. hIL-13Rα2.Fc Fusion Polypeptide Co-Expressed with hIL-13 is Stable to Cold-Storage

Purified hIL-13Rα2.Fc fusion polypeptide dimer has been shown to be susceptible to forming high molecular weight aggregates upon storage. The effect of a 6-day cold-storage (4° C.) on the molecular aggregation state of hIL-13Rα2.Fc fusion polypeptide obtained from 37A4 cells co-expressing hIL-13 and hIL-13Rα2.Fc fusion polypeptide was compared with the effect of cold-storage on the molecular aggregation of hIL-13Rα2.Fc fusion polypeptide produced by the 6FD3 parental cell line using SEC-HPLC. Briefly, Protein A purified hIL-13Rα2.Fc fusion polypeptide from 6FD3 parent cell line or IL-13 co-expressing cell line 37A4 was held at 4° C. for 6 days. The material was analyzed by SEC-HPLC on day 0, day 3, and day 6.

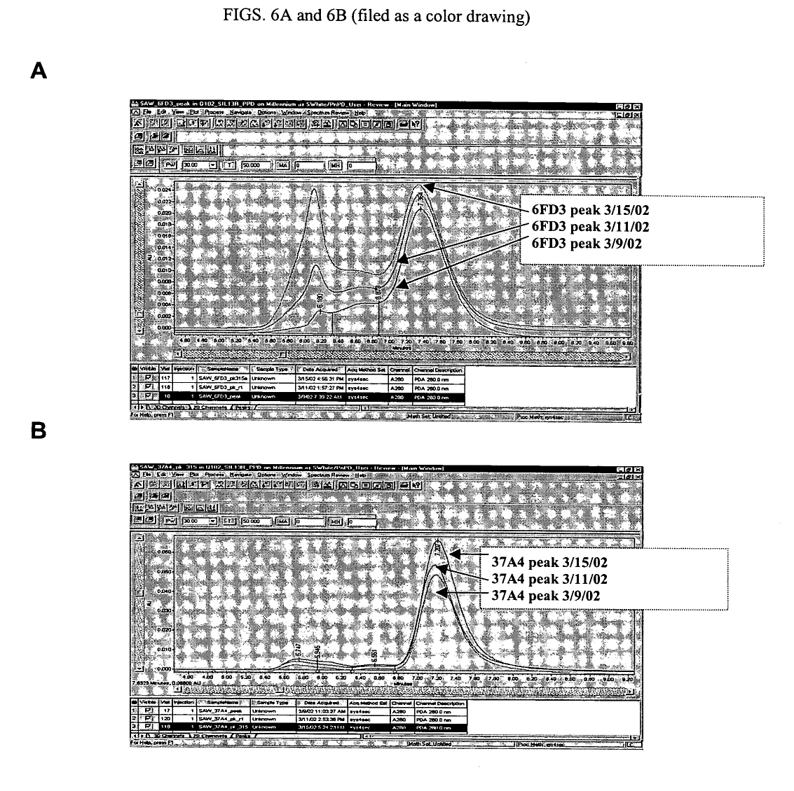

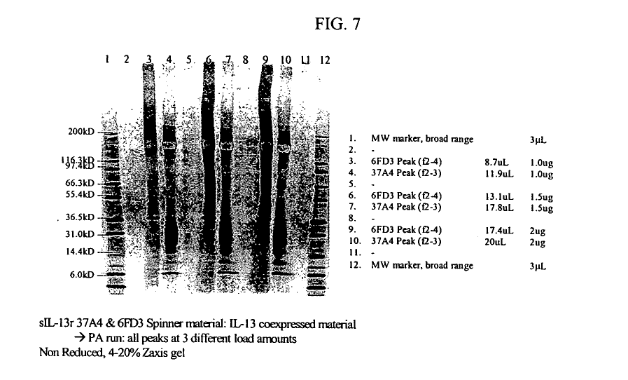

Chromatographs were overlaid to show the relative distribution of the major hIL-13Rα2.Fc fusion polypeptide species (FIG. 6).

As shown in FIG. 6A, the HMW1 and HMW2 peaks increase over time in the material produced from the 6FD3 parent cell line. In contrast, FIG. 6B shows that the HMW1 and HMW2 peaks remain low in the hIL-13Rα2.Fc fusion polypeptide-containing material made in the 37A4 hIL-13 co-expressing cell line.

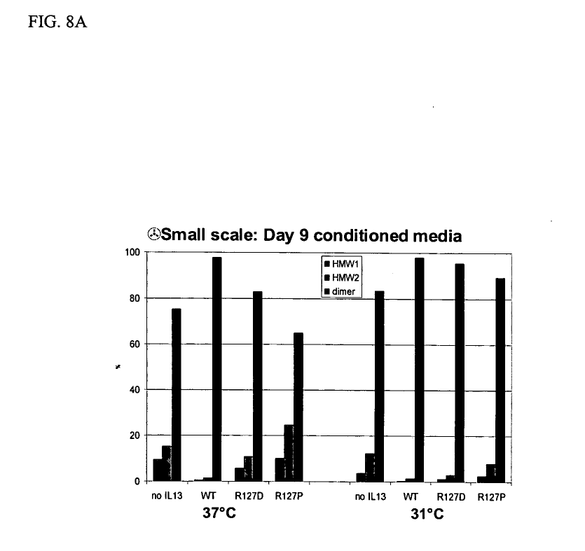

The protein A purified material from 6FD3 parent cell line or 37A4 hIL-13 co-expressing cell line was also analyzed by SDS-PAGE (4-20% acrylamide gradient gel, subsequently silver stained). As shown in FIG. 7, fewer contaminating bands were observed in the material made in the co-expressing cell line as compared with the parent cell line. These results are consistent with data obtained using size exclusion chromatography.

### Example 4

### Cells that Coexpress Mutant Forms of hIL-13 (R127D or R127P) with hIL-13Rα2.FC Fusion Polypeptide Decreased Levels of the Fusion Polypeptide

p The amount of fusion hIL-13Rα2.Fc fusion polypeptide expressed following co-expression with wild-type or mutant forms of HL-13 was examined. Mutant forms tested included hIL-13R127D and R127P. Expression was determined at both 31° C. and 37° C.

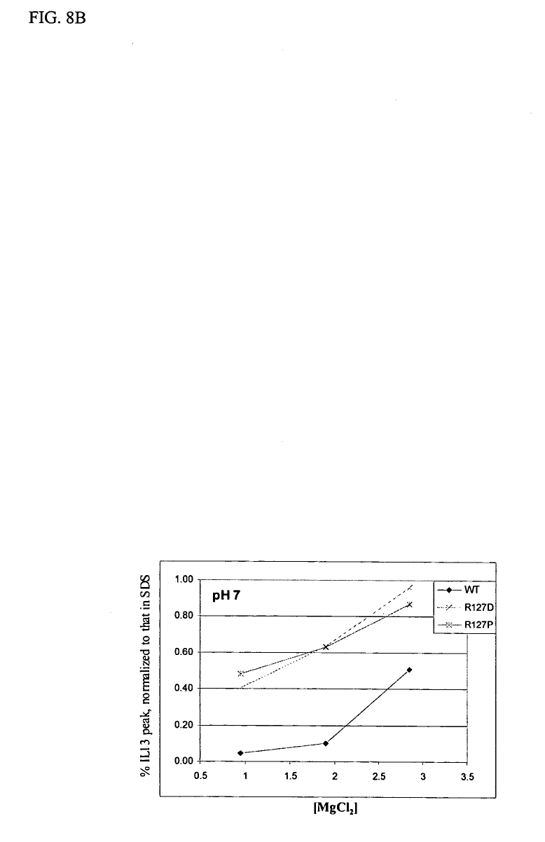

The results of coexpressing of hIL-13Rα2.Fc fusion polypeptide with wild-type or mutant hIL-13 at 37° C. or 31° C. are shown in FIG. 8A. Cells expressing only the hIL-13Rα2.Fc fusion polypeptide showed high levels of aggregate when cultured at both 37° C. or 31° C. (“no IL13”). Cells coexpressing wild-type hIL-13 with hIL-13Rα2.Fc fusion polypeptide exhibit reduced levels of aggregate at both culture temperatures. Cells that coexpressed mutant forms of hIL-13 (R127D or R127P) with hIL-13Rα2.Fc fusion polypeptide exhibit decreased levels of the fusion polypeptide only at the lower culture temperature in these experiments.

The ability of hIL-13Rα2.Fc to dissociate from a coexpressed wild-type, R127D or R127P IL-13 ligand was next examined. Dissociation was assessed by determining the ability of MgCl2 to dissociate IL-13 from a IL-13-hIL-13Rα2.Fc complex. Conditioned media from cells coexpressing hIL-13Rα2.Fc fusion polypeptide with wild-type or mutant hIL-13 was purified on a Protein A column in the presence of increasing concentrations of MgCl₂. The amount of dissociated IL-13 at each MgCl₂ concentration was then measured.

The results are shown in FIG. 8B. The graph shows the hIL-13 peak area on an SEC-HPLC chromatograph, normalized to the hIL-13 peak when the complex is completely dissociated by SDS at varying concentrations of MgCl₂ Wash buffer with increasing levels of MgCl₂ could efficiently dissociate the mutant, but not wild-type, hIL-13 polypeptide from the hIL-13Rα2.Fc fusion polypeptide.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of producing an interleukin-13 (IL-13) antagonist polypeptide, the method comprising:
providing a culture medium comprising a host cell, wherein said host cell expresses a nucleic acid encoding said IL-13 antagonist polypeptide and said host cell expresses a nucleic acid encoding a complexing polypeptide for said IL-13 antagonist polypeptide;
culturing said host cell under conditions allowing for expression of said IL-13 antagonist polypeptide and said complexing polypeptide; and
recovering said IL-13 antagonist polypeptide from said culture medium, thereby producing said IL-13 antagonist polypeptide.

2. The method of claim 1, wherein said complexing polypeptide is IL-13.

3. The method of claim 1, wherein said complexing polypeptide comprises the amino acid sequence of a human IL-13 polypeptide of SEQ ID NO:17 or comprises a variant amino acid sequence of SEQ ID NO:17 wherein the arginine at amino acid 126 is replaced with aspartic acid, glutamic acid, or proline.

4. The method of claim 1, wherein said complexing polypeptide is IL-6.

5. The method of claim 1, wherein said nucleic acid encoding said IL-13 antagonist polypeptide is an exogenous nucleic acid for said host cell.

6. The method of claim 5, further comprising introducing said exogenous nucleic acid into said host cell.

7. The method of claim 1, wherein said nucleic acid encoding said complexing polypeptide is an exogenous nucleic acid.

8. The method of claim 7, further comprising introducing said exogenous nucleic acid into said host cell.

9. The method of claim 1, wherein more IL-13 antagonist polypeptide is recovered when said IL-13 antagonist polypeptide is co-expressed with said complexing polypeptide than when said IL-13 antagonist polypeptide is expressed in the absence of said complexing polypeptide.

10. The method of claim 1, wherein said host cell is cultured at a temperature of from about 29° C. to about 39° C. when expressing said nucleic acid encoding said IL-13 antagonist polypeptide and said complexing polypeptide.

11. The method of claim 1, wherein said expression of said IL-13 antagonist polypeptide in said host cell is conducted at a temperature of about 31° C. when expressing said nucleic acid encoding said IL-13 antagonist polypeptide and said complexing polypeptide.

12. The method of claim 1, wherein said expression of said IL-13 antagonist polypeptide in said host cell is conducted at a temperature of about 37° C. when expressing said nucleic acid encoding said IL-13 antagonist polypeptide and said complexing polypeptide.

13. The method of claim 1, wherein said host cell is a stably transfected cell.

14. The method of claim 1, wherein said host cell is a Chinese Hamster Ovary (CHO) cell.

15. The method of claim 1, wherein said host cell is a transiently transfected cell.

16. The method of claim 15, wherein said host cell is a COS cell.

17. The method of claim 1, wherein said IL-13 antagonist polypeptide includes an extracellular moiety of an IL-13 receptor polypeptide fused to at least a portion of an immunoglobulin polypeptide.

18. The method of claim 17, wherein said IL-13 receptor polypeptide is an IL-13Rα2 polypeptide.

19. The method of claim 18, wherein said IL-13 antagonist polypeptide includes an Fc region of an immunoglobulin γ1 polypeptide.

20. The method of claim 19, wherein said IL-13 antagonist polypeptide is IL-13 Rα.2Fc.

21. The method of claim 1, wherein said complexing polypeptide for said IL-13 antagonist polypeptide is an IL-13 receptor binding fragment of an IL-13 polypeptide.

22. The method of claim 1, wherein said complexing polypeptide for said IL-13 antagonist polypeptide comprises the amino acid sequence of a non-naturally occurring IL-13 polypeptide.

23. The method of claim 1, wherein said complexing polypeptide for said IL-13 antagonist polypeptide is an antibody to an IL-13 receptor polypeptide.

24. The method of claim 1, wherein aggregation of said expressed IL-13 antagonist polypeptide is reduced relative to aggregation of said IL-13 antagonist polypeptide expressed in a host cell not expressing said nucleic acid encoding said complexing polypeptide for said IL-13 polypeptide.

25. The method of claim 24, wherein aggregation of said expressed IL-13 antagonist polypeptide is reduced at least about 10% relative to aggregation of said IL-13 antagonist polypeptide expressed in a host cell not expressing said nucleic acid encoding said complexing polypeptide for said IL-13 polypeptide.

26. The method of claim 24, wherein aggregation of said expressed IL-13 antagonist polypeptide is reduced at least about 30% relative to aggregation of said IL-13 antagonist polypeptide expressed in a host cell not expressing said nucleic acid encoding said complexing polypeptide for said IL-13 polypeptide.

27. The method of claim 24, wherein aggregation of said expressed IL-13 antagonist polypeptide is reduced at least about 90% relative to aggregation of said IL-13 antagonist polypeptide expressed in a host cell not expressing said nucleic acid encoding said complexing polypeptide for said IL-13 polypeptide.

28. A pharmaceutical composition comprising said IL-13 antagonist polypeptide produced by the method of claim 1 and a pharmaceutically acceptable carrier.

29. A method of reducing the level of IL-13 in a patient comprising administering to said patient a therapeutically effective amount of the composition of claim 28.

30. A method of producing an IL-13 Rα2.Fc polypeptide, the method comprising:
providing a culture medium comprising a cell, wherein said cell expresses a nucleic acid encoding IL-13 Rα2.Fc polypeptide and said cell expresses a nucleic acid encoding a complexing polypeptide for said IL-13 Rα2.Fc polypeptide;
culturing said cell under conditions allowing for expression of said IL-13 Rα2.Fc polypeptide and said complexing polypeptide; and
recovering said IL-13 Rα2.Fc polypeptide from said culture medium, thereby producing said IL-13 Rα2.Fc polypeptide.

31. A method of producing an IL-13 Rα2.Fc polypeptide, the method comprising:
providing a culture medium comprising a cell, wherein said cell expresses a nucleic acid encoding said IL-13 Rα2.Fc polypeptide and said cell expresses a nucleic acid encoding an IL-13 polypeptide;
culturing said cell under conditions allowing for expression of said IL-13 Rα2.Fc polypeptide and said IL-13 polypeptide; and
recovering said IL-13 Rα2.Fc polypeptide from said culture medium, thereby producing said IL-13 Rα2.Fc polypeptide.

32. The method of claim 1, wherein more IL-13 Rα2.Fc polypeptide is recovered when said IL-13 Rα2.Fc polypeptide is co-expressed with IL-13 than when said IL-13 Rα2.Fc polypeptide is expressed in the absence of IL-13.

33. A pharmaceutical composition comprising said IL-13 Rα2.Fc polypeptide produced by the method of claim 31 and a pharmaceutically acceptable carrier.

34. A method of reducing the level of a cytokine in a patient comprising administering to said patient a therapeutically effective amount of the composition of claim 33.

35. A purified preparation of a soluble IL-13 antagonist polypeptide, wherein at least 40% of said soluble IL-13 antagonist polypeptide is present in monomer or dimer form following incubation for at least one week at 4° C.

36. The preparation of claim 35, wherein at least 60% of said soluble IL-13 antagonist polypeptide is present in monomer or dimer form following incubation for at least one week at 4° C.

37. The preparation of claim 35, wherein at least 80% of said soluble IL-13 antagonist polypeptide is present in monomer or dimer form following incubation for at least one week at 4° C.

38. The preparation of claim 35, wherein at least 90% of said soluble IL-13 antagonist polypeptide is present in monomer or dimer form following incubation for at least one week at 4° C.
